(19) United States
(12) Patent Application Publication
(10) Pub. No.: US 2004/0053959 A1
(43) Pub. Date: Mar. 18, 2004

(54) Novel processes and intermediates for preparing triazolo-pyridines

(75) Inventors: Richard A. Buzon SR., Stonington, CT (US); Michael J. Castaldi, Pawcatuck, CT (US); Zhengong B. Li, East Lyme, CT (US); David H. B. Ripin, Old Saybrook, CT (US); Yong Tao, Salem, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/649,247

(22) Filed: Aug. 27, 2003

Related U.S. Application Data
(60) Provisional application No. 60/407,085, filed on Aug. 30, 2002.

(30) Foreign Application Priority Data
Jul. 13, 2000 (WO) WO 00/40243
Oct. 26, 2000 (WO) WO 00/63204
Jun. 2, 2000 (WO) WO 00/31065
Feb. 10, 2000 (WO) WO 00/06563
Jul. 20, 2000 (WO) WO 00/41698

(51) Int. Cl. C07D 471/02

(52) U.S. Cl. 514/303; 546/119

(57) ABSTRACT

The present invention relates and intermediates to a novel process for preparing triazolo-pyridines of the formula I

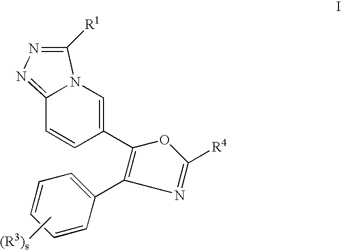

wherein R¹ is selected from the group consisting of hydrogen, (C₁-C₆)alkyl or other suitable substituents;

R³ is selected from the group consisting of hydrogen, (C₁-C₆)alkyl or other suitable substituents;

s is an integer from 0-5;

R⁴ is hydrogen or a suitable substituent and to intermediates for their preparation. The compounds prepared by the methods of the present invention are potent inhibitors of MAP kinases, preferably p38 kinase. They are useful in the treatment of inflammation, osteoarthritis, rheumatoid arthritis, cancer, reperfusion or ischemia in stroke or heart attack, autoimmune diseases and other disorders.

The present invention relates to novel processes for preparing triazolo-pyridines, to intermediates useful in their preparation. The compounds that can be prepared by the methods of the invention are potent inhibitors of MAP kinases, preferably p38 kinase (MAPK14/CSBP/RK kinase). The compounds that can be prepared by the methods of the invention are therefore useful in the treatment of inflammation, osteoarthritis, rheumatoid arthritis, cancer, reperfusion or ischemia in stroke or heart attack, autoimmune diseases and other disorders.

MAP kinases and MAPK14/CSBP/p38/RK kinase inhibitors are well known to those skilled in the art. U.S. Provisional Applications 60/274791, 60/274840 and 60/281331, filed Mar. 9, 2001, Mar. 9, 2001 and Apr. 4, 2001, respectively, and entitled “Novel Antiinflammatory Compounds,” “Novel Triazolopyridine Antiinflammatory Compounds” and “Novel Benzotriazole Antiinflammatory Compounds,” respectively, refer to certain inhibitors of MAP kinases, preferably p38 kinase. International Patent Publication WO 00/40243, published Jul. 13, 2000, refers to pyridine substituted pyridine compounds and states that these compounds are p38 inhibitors. International Patent Publication WO 00/65004, published Oct. 26, 2000, refers to substituted azole compounds and states that these compounds are p38 inhibitors. International Patent Publication WO 00/31065, published Jun. 2, 2000, refers to certain heterocyclic compounds and states that these compounds are p38 inhibitors. International Patent Publication WO 00/06563, published Feb. 10, 2000, refers to substituted imidazole compounds and states that these compounds are p38 inhibitors. International Patent Publication WO 00/41698, published Jul. 20, 2000, refers to certain ω-carboxy aryl substituted diphenyl urea compounds and states that these compounds are p38 inhibitors. U.S. Pat. No. 6,288,062 refers to certain substituted oxazole compounds and states that these compounds are p38 inhibitors. U.S. Pat. No. 5,716,955 refers to certain substituted imidazole compounds and states that these compounds are p38 inhibitors. U.S. Pat. No. 5,716,972 refers to certain pyridinyl substituted imidazole compounds and states that these compounds are p38 inhibitors. U.S. Pat. No. 5,756,499 refers to certain substituted imidazole compounds and states that these compounds are p38 inhibitors.

### SUMMARY OF THE INVENTION

The present invention relates to a process for preparing a compound of the formula

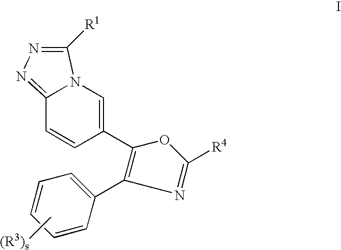

wherein R¹ is selected from the group consisting of hydrogen, —C≡N, (C_{1-C}₆)alkyl, (C₂-C₆)alkenyl, (C₂-C₆)alkynyl, (C₃-C₁₀)cycloalkyl, phenyl, (C₁-C₁₀)heteroaryl, (C₁-C₁₀)heterocyclic and (R²)₂—N—; wherein each of the aforesaid (C₁-C₆)alkyl, (C₃-C₁₀)cycloalkyl, phenyl, (C₁-C₁₀)heteroaryl and (C₁-C₁₀)heterocyclic substituents may optionally be independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, (C₁-C₆)alkyl-O—(C═O)—, [(C₁-C₆)alkyl]₂-N—(C═O)—, phenyl-[((C₁-C₆)alkyl)-N]—(C═O)—, —NO₂, [(C₁-C₆)alkyl]₂-amino, (C₁-C₆)alkyl-(C═O)—[((C₁-C₆)alkyl)-N]—, phenyl-(C═O)—[((C₁-C₆)alkyl)-N]—, [(C₁-C₆)alkyl-]₂N—(C═O)—[((C₁-C₆)alkyl)-N]—, (phenyl-)₂N—(C═O)—[((C₁-C₆)alkyl)-N]—, (C₁-C₆)alkyl-O—(C═O)—[((C₁-C₆)alkyl)-N]—, phenyl-O—(C═O)—[((C₁-C₆)alkyl)-N]—, (C₁-C₆)alkyl-SO₂—, phenyl-SO₂—, (C₁-C₆)alkoxy, perhalo(C₁-C₆)alkoxy, phenoxy, (C₁-C₆)alkyl-(C═O)—O—, phenyl-(C═O)—O—, [(C₁-C₆)alkyl-]₂N—(C═O)—O—, (phenyl-)₂N—(C═O)—O—; wherein when said R² phenyl contains two adjacent substituents, such substituents may optionally be taken together with the carbon atoms to which they are attached to form a five to six membered carbocyclic or heterocyclic ring; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy;

each R² is independently selected from hydrogen, (C₁-C₆)alkyl, phenyl, (C₁-C₁₀)heteroaryl, (C₁-C₁₀)heterocyclic and (C₃-C₁₀)cycloalkyl; wherein each of the aforesaid R² (C₁-C₆)alkyl, phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, —NO₂, [(C₁-C₆)alkyl]₂-amino, (C₁-C₆)alkyl-(C═O)—[((C₁-C₆)alkyl)-N]—, phenyl-(C═O)—[((C₁-C₆)alkyl)-N]—, —CN, (C₁-C₆)alkyl-(C═O)—, phenyl-(C ═O)—, (C₁-C₁₀)heteroaryl-(C═O)—, (C₁-C₁₀)heterocyclic-(C═O)—, (C₃-C₁₀)cycloalkyl-(C═O)—, (C₁-C₆)alkyl-O—(C═O)—, [(C₁-C₆)alkyl]₂-N—(C═O)—, phenyl-[((C₁-C₆)alkyl)-N]—(C═O)—, (C₁-C₆)alkyl-(C═O)—O— and phenyl-(C═O)—O—; wherein two R² (C₁-C₆)alkyl groups may be taken together with the nitrogen atom to which they are attached to form a five to six membered heterocyclic or heteroaryl ring;

each R³ is independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)— and (C₁-C₆)alkyl-(C═O)—O—; wherein two adjacent R³ substituents may be optionally taken together with the carbon atoms to which they are attached to form a five to six membered carbocyclic or heterocyclic ring;

s is an integer from zero to five;

R⁴ is selected from the group consisting of hydrogen, fluoro, chloro or R⁵—B—(CH₂)ₙ—;

n is an integer from zero to six;

each B is independently a bond, —(CHR⁶)—, —O—, —S—, —(SO₂)—, —(C═O)—, —O—(C═O)—, —(C═O)—O—, —(C═O)—NR⁶—, —(R⁶—N)—, —(R⁶—N)—SO₂—, —(R⁶—N)—(C═O)—, —SO₂—(NR⁶)—, —(R⁶—N )—(C═O)—(NR⁷)—, —(O)—(C═O)—(NR⁶)— or —(R⁶—N)—(C═O)—O—;

R⁵ is selected from the group consisting of hydrogen, —CF₃, —C≡N, R⁹—(R⁸CH)ₘ—, phenyl, (C₁-C₁₀)heterocyclic, (C₁-C₁₀)heteroaryl, and (C₃-C₁₀)cycloalkyl; wherein each of the aforesaid R⁵ phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—; wherein two adjacent R⁵ substituents of said phenyl, (C₁-C₁₀)heteroaryl, (C₁-C₁₀)heterocyclic and (C₃-C₁₀)cycloalkyl may optionally be taken together with the carbon or heteroatom to which they are attached to form a five or six membered carbocyclic or heterocyclic ring;

m is an integer from one to six;

R⁶ is hydrogen, (C₁-C₆)alkyl-SO₂— or (C₁-C₆)alkyl;

R⁷ is hydrogen or (C₁-C₆)alkyl;

each R⁸ is independently selected from the group consisting of hydrogen, amino, (C₁-C₆)alkoxy and (C₁-C₆)alkyl;

R⁹ is selected from the group consisting of hydrogen, (C₁-C₆)alkyl, (C₂-C₆)alkenyl, (C₂-C₆)alkynyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, phenyl-SO₂—NH—, (C₁-C₆)alkyl-SO₂—[((C₁-C₆)alkyl)-N]—, phenyl-SO₂—[((C₁-C₆)alkyl)-N]—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—;

or an acceptable salt thereof; comprising reacting a compound of the formula

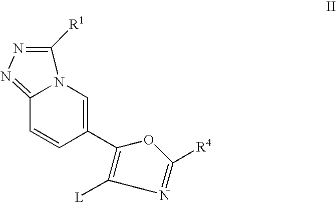

wherein L is a leaving group and R¹ and R⁴ are as defined above, with a compound of the formula

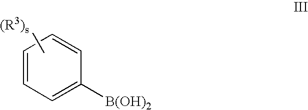

wherein R³ and s are as defined above and a transition metal catalyst (such as a palladium catalyst, such as palladium acetate (Pd(OAc)₂), tetrakis (triphenylphosphine) palladium (0), palladium tetra-triphenylphosphine (Pd(PPh₃)₄), Pd(dppf)Cl₂, tris(dibenzylidene acetone)dipalladium(0) (Pd₂(dba)₃), and di(dibenzylidene acetone) palladium(0) (Pd(dba)₂)). Preferably, the reaction is done in the presence of toluene, including mixtures thereof.

The present invention also relates to a process for preparing a compound of the formula

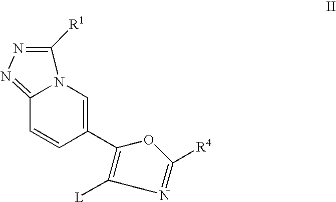

wherein L is a leaving group;

R¹ is selected from the group consisting of hydrogen, —C≡N, (C₁-C₆)alkyl, (C₂-C₆)alkenyl, (C₂-C₆)alkynyl, (C₃-C₁₀)cycloalkyl, phenyl, (C₁-C₁₀)heteroaryl, (C₁-C₁₀)heterocyclic and (R²)₂—N—; wherein each of the aforesaid (C₁-C₆)alkyl, (C₃-C₁₀)cycloalkyl, phenyl, (C₁-C₁₀)heteroaryl and (C₁-C₁₀)heterocyclic substituents may optionally be independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, —NO₂, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, phenyl-HN—(C═O)—NH—, (phenyl-)₂N—(C═O)—NH—, phenyl-HN—(C═O)—[((C₁-C₆)alkyl)-N]—, (phenyl-)₂N—(C═O)—[((C₁-C₆)alkyl)-N]—, (C₁-C₆)alkyl-O—(C═O)—NH—, (C₁-C₆)alkyl-O—(C═O)—[((C₁-C₆)alkyl)-N]—, phenyl-O—(C═O)—NH—, phenyl-O—(C═O)—[((C₁-C₆)alkyl)-N]—, (C₁-C₆)alkyl-SO₂NH—, phenyl-SO₂NH—, (C₁-C₆)alkyl-SO₂—, phenyl-SO₂—, hydroxy, (C₁-C₆)alkoxy, perhalo(C₁-C₆)alkoxy, phenoxy, (C₁-C₆)alkyl-(C═O)—O—, phenyl-(C═O)—O—, H₂N—(C═O)—O—, (C₁-C₆)alkyl-HN—(C═O)—O—, [(C₁-C₆)alkyl-]₂N—(C═O)—O—, phenyl-HN—(C═O)—O—, (phenyl-)₂N—(C═O)—O—; wherein when said R¹ phenyl contains two adjacent substituents, such substituents may optionally be taken together with the carbon atoms to which they are attached to form a five to six membered carbocyclic or heterocyclic ring; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy;

each R² is independently selected from hydrogen, (C₁-C₆)alkyl, phenyl, (C₁-C₁₀)heteroaryl, (C₁-C₁₀)heterocyclic and (C₃-C₁₀)cycloalkyl; wherein each of the aforesaid R² (C₁-C₆)alkyl, phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—; wherein two R² (C₁-C₆)alkyl groups may be taken together with the nitrogen atom to which they are attached to form a five to six membered heterocyclic or heteroaryl ring;

R⁴ is selected from the group consisting of hydrogen, fluoro, chloro or R⁵—B—(CH₂)ₙ—;

n is an integer from zero to six;

each B is independently a bond, —(CHR⁶)—, —O—, —S—, —(SO₂)—, —(C═O)—, —O—(C═O)—, —(C═O)—O—, —(C═O)—NR⁶—, —(R⁶—N)—, —(R⁶—N)—SO₂—, —(R⁶—N)—(C═O)—, —SO₂—(NR⁶)—, —(R⁶—N)—(C═O)—(NR⁷)—, —(O)—(C═O)—(NR⁶)— or —(R⁶—N)—(C═O)—O—;

R⁵ is selected from the group consisting of hydrogen, —CF₃, —C≡N, R⁹—(R⁸CH)ₘ—, phenyl, (C₁-C₁₀)heterocyclic, (C₁-C₁₀)heteroaryl, and (C₃-C₁₀)cycloalkyl; wherein each of the aforesaid R⁵ phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—; wherein two adjacent R⁵ substituents of said phenyl, (C₁-C₁₀)heteroaryl, (C₁-C₁₀)heterocyclic and (C₃-C₁₀)cycloalkyl may optionally be taken together with the carbon or heteroatom to which they are attached to form a five or six membered carbocyclic or heterocyclic ring;

m is an integer from one to six;

R⁶ is hydrogen, (C₁-C₆)alkyl-SO₂— or (C₁-C₆)alkyl;

R⁷ is hydrogen or (C₁-C₆)alkyl;

each R⁸ is independently selected from the group consisting of hydrogen, amino, (C₁-C₆)alkoxy and (C₁-C₆)alkyl;

R⁹ is selected from the group consisting of hydrogen, (C₁-C₆)alkyl, (C₂-C₆)alkenyl, (C₂-C₆)alkynyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, phenyl-SO₂—H—(C₁-C₆)alkyl-SO₂—[((C₁-C₆)alkyl)-N]—, phenyl-SO₂—[((C₁-C₆)alkyl)-N]—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—;

by reaction of a compound of the formula

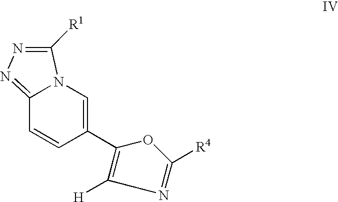

wherein R¹ and R⁴ are as defined above; with a strong base (such as lithium diisopropylamide, lithium bis(trimethylsilyl)amide, lithium dialkylamines, alkyl lithiums (such as n-butyllithium, sec-butyllithium or tert-butyllithium), and aryl lithiums (such as benzyl lithium), preferably lithium diisopropylamide, lithium bis(trimethylsilyl)amide or lithium dialkylamines, a halogenating reagent (such as phenyl trimethylammonium tribromide, N-bromosuccinimide, pyridinium bromide, perbromide, Br₂, Br₂—Ph₃P, MBS, N-iodosuccinimide or I₂) and a polar aprotic solvent (such as N,N-dimethylformamide (DMF), N,N-dimethylacetamide (DMAC) or N-methylpyrrolidinone (NMP), 1,3 dimethylimidazolidinone (DMI), 1,3-dimethyl-3,4,5,6-tetrahydro-2(1H)-pyrimidinone (DMPU) and combinations thereof).

The present invention also relates to a process for preparing a compound of the formula

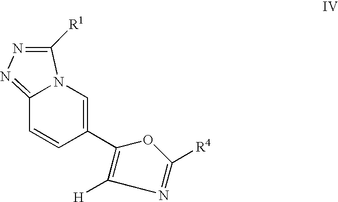

wherein R⁴ is hydrogen and R¹ is as defined above in for the compound of formula l; comprising reacting a compound of the formula

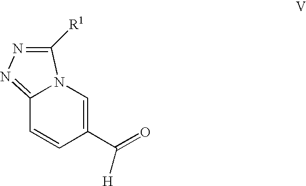

wherein R¹ is as defined above; with tosylmethyl isocyanide and a base (such as potassium carbonate, potassium t-butoxide, sodium bicarbonate, triethylamine, or dimethylaminopyridine).

The present invention also relates to a process for preparing a compound of the formula

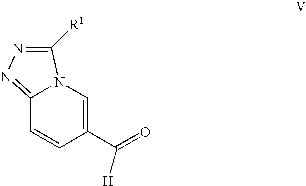

wherein R¹ is as defined above in claim 2; by reaction of a compound of the formula

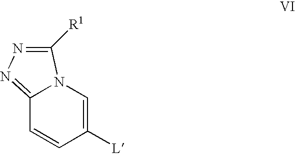

wherein L′ is bromo or iodo and R′ is as defined above; with an (C₁-C₆)alkyl magnesium halide or (C₁-C₆)alkyl lithium, followed by reaction with a disubstituted formamide reagent. Preferably the work-up of the aforesaid reaction is done in the absence of a strong acid or base, preferably in the presence of a weak acid such as aqueous citric acid or potassium dihydrogen phosphate.

The present invention also relates to a process for preparing a compound of the formula

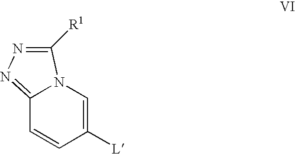

wherein L′ is halo;

R¹ is selected from the group consisting of hydrogen, —C≡N, (C₁-C₆)alkyl, (C₂-C₆)alkenyl, (C₂-C₆)alkynyl, (C₃-C₁₀)cycloalkyl, phenyl, (C₁-C₁₀)heteroaryl, (C₁-C₁₀)heterocyclic and (R²)₂—N—; wherein each of the aforesaid (C₁-C₆)alkyl, (C₃-C₁₀)cycloalkyl, phenyl, (C₁-C₁₀)heteroaryl and (C₁-C₁₀)heterocyclic substituents may optionally be independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, (C₁-C₆)alkyl-O—(C═O)—, [(C₁-C₆)alkyl]₂-N—(C═O)—, phenyl-[((C₁-C₆)alkyl)-N)—(C═O)—, —NO₂, [(C₁-C₆)alkyl]₂-amino, (C₁-C₆)alkyl-(C═O)—[((C₁-C₆)alkyl)-N]—, phenyl-(C═O)—[((C₁-C₆)alkyl)-N]—, [(C₁-C₆)alkyl-]₂N—(C═O)—[((C₁-C₆)alkyl)-N]—, (phenyl-)₂N—(C═O)—[((C₁-C₆)alkyl)-N]—, (C₁-C₆)alkyl-O—(C═O)—[((C₁-C₆)alkyl)-N]—, phenyl-O—(C═O)—[((C₁-C₆)alkyl)-N]—, (C₁-C₆)alkyl-SO₂—, phenyl-SO₂—, (C₁-C₆)alkoxy, perhalo(C₁-C₆)alkoxy, phenoxy, (C₁-C₆)alkyl-(C═O)—O—, phenyl-(C═O)—O—, [(C₁-C₆)alkyl-]₂N—(C═O)—O—, (phenyl-)₂N—(C═O)—O—; wherein when said R¹ phenyl contains two adjacent substituents, such substituents may optionally be taken together with the carbon atoms to which they are attached to form a five to six membered carbocyclic or heterocyclic ring; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy;

and each R² is independently selected from hydrogen, (C₁-C₆)alkyl, phenyl, (C₁-C₁₀)heteroaryl, (C₁-C₁₀)heterocyclic and (C₃-C₁₀)cycloalkyl; wherein each of the aforesaid R² (C₁-C₆)alkyl, phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, —NO₂, [(C₁-C₆)alkyl]₂-amino, (C₁-C₆)alkyl-(C═O)—[((C₁-C₆)alkyl)-N]—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, (C₁-C₆)alkyl-O—(C═O)—, [(C₁-C₆)alkyl]₂-N—(C═O)—, phenyl-[((C₁-C₆)alkyl)-N]—(C═O)—, (C₁-C₆)alkyl-(C═O)—O— and phenyl-(C═O)—O—; wherein two R² (C₁-C₆)alkyl groups may be taken together with the nitrogen atom to which they are attached to form a five to six membered heterocyclic or heteroaryl ring;

comprising reacting a compound of the formula

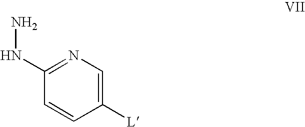

wherein L′ is halo; with a reagent (such as an acid anhydride or an acid chloride) of the formula

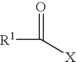

wherein X is halo, tosyl, mesyl or a group of the formula

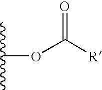

wherein R′ is R¹, t-butyl, or (C₁-C₆)alkyl-O—.

More specifically, the aforesaid reaction also relates to a process wherein R¹ is isopropyl and said reagent is isobutyryl chloride.

More specifically, the aforesaid reaction also relates to a process wherein R¹ is other than isopropyl and said reagent is a compound of the formula

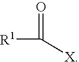

The present invention also relates to a process for preparing a compound of the formula

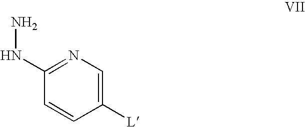

wherein L′ is halo;

comprising reacting a compound of the formula

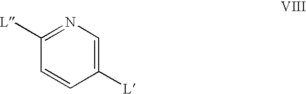

wherein L′ is halo and L″ is halo; with a hydrazine, PEG-300, water and 2-butanol.

The present invention also relates to a process for preparing a compound of the formula

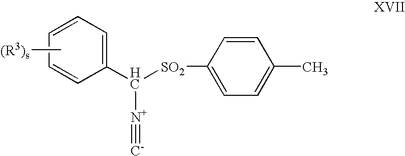

wherein each R³ is independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)— and (C₁-C₆)alkyl-(C═O)—O—; wherein two adjacent R³ substituents may be optionally taken together with the carbon atoms to which they are attached to form a five to six membered carbocyclic or heterocyclic ring;

s is an integer from zero to five;

or an acceptable salt thereof; comprising reacting a compound of the formula

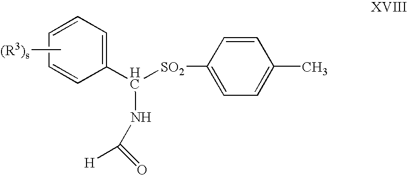

wherein R³ and s are as defined above, in the presence of a dehydrating agent such as POCl₃, and a weak hindered base such as 2,6 lutidine or 2,4,6 trimethyl pyridine. Preferably the reaction is performed in the presence of a solvent such as tetrahydrofuran, dimethyl ether or methylene chloride.

An embodiment of the present invention are those compounds of formula I wherein R² is (C₁-C₆)alkyl, phenyl, (C₃-C₁₀)cycloalkyl, (C₁-C₁₀)heteroaryl or (C₁-C₁₀)heterocyclic.

Another embodiment of the present invention are those compounds of formula I wherein R¹ is (C₁-C₆)alkyl, optionally substituted with one to four groups independently selected from halo, hydroxy, (C₁-C₆)alkyl, (C₂-C₆)alkenyl, (C₂-C₆)alkynyl, (C₁-C₆)alkoxy, perhalo(C₁-C₆)alkyl, perhalo(C₁-C₆)alkoxy, —CN, —NO₂, amino, (C₁-C₆)alkylamino, [(C₁-C₆)alkyl]₂-amino, HO—(C═O)—, (C₁-C₆)alkyl-(C═O)—, (C₁-C₆)alkyl-O—(C═O)—, (C₁-C₆)alkyl-CO₂—, (C₁-C₆)alkyl-(C═O)—NH—, (C₁-C₆)alkyl-NH—(C═O)—, (C₁-C₆)alkyl-(C═O)—[((C₁-C₆)alkyl)-N]—, (C₁-C₆)alkyl-[((C₁-C₆)alkyl)-N]—(C═O)—, (C₁-C₆)alkyl-SO₂NH—, (C₁-C₆)alkyl-SO₂—, optionally substituted phenyl-(C═O)—, optionally substituted phenyl-(C═O)—O—, optionally substituted phenoxy, optionally substituted phenyl-NH—(C═O)—, optionally substituted phenyl-[((C₁-C₆)alkyl)-N]—(C═O)—, optionally substituted phenyl-(C═O)—NH— optionally substituted phenyl-(C═O)—[((C₁-C₆)alkyl)-N]—.

A preferred embodiment of the present invention refers to those methods wherein R¹ is (C₁-C₄)alkyl.

Another embodiment of the present invention refers to those methods wherein R¹ is optionally substituted (C₃-C₆)cycloalkyl; wherein said substituents are independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, —NO₂, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, phenyl-HN—(C═O)—NH—, (phenyl-)₂N—(C═O)—NH—, phenyl-HN—(C═O)—[((C₁-C₆)alkyl)-N]—, (phenyl-)₂N—(C═O)—[((C₁-C₆)alkyl)-N]—, (C₁-C₆)alkyl-O—(C═O)—NH—, (C₁-C₆)alkyl-O—(C═O)—[((C₁-C₆)alkyl)-N]—, phenyl-O—(C═O)—NH—, phenyl-O—(C═O)—[((C₁-C₆)alkyl)-N]—, (C₁-C₆)alkyl-SO₂NH—, phenyl-SO₂NH—, (C₁-C₆)alkyl-SO₂—, phenyl-SO₂-, hydroxy, (C₁-C₆)alkoxy, perhalo(C₁-C₆)alkoxy, phenoxy, (C₁-C₆)alkyl-(C═O)—O—, phenyl-(C═O)—O—, H₂N—(C═O)—O—, (C₁-C₆)alkyl-HN—(C═O)—O—, [(C₁-C₆)alkyl-]₂N—(C═O)—O—, phenyl-HN—(C═O)—O—, (phenyl-)₂N—(C═O)—O—; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy; more preferably said substituents are independently selected from the group consisting of halo, (C₁-C₆)alkyl, (C₂-C₆)alkenyl, perhalo(C₁-C₆)alkyl, —CN, (C₁-C₆)alkyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, hydroxy, (C₁-C₆)alkoxy, perhalo(C₁-C₆)alkoxy, (C₁-C₆)alkyl-(C═O)—O—, H₂N—(C═O)—O—, (C₁-C₆)alkyl-HN—(C═O)—O— and [(C₁-C₆)alkyl-]₂N—(C═O)—O—.

Another embodiment of the present invention refers to those methods wherein R¹ is optionally substituted (C₁-C₁₀)heterocyclic; wherein said substituents are independently selected from the group consisting of halo, (C₁-C₆)alkyl, (C₂-C₆)alkenyl, (C₂-C₈)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, —NO₂, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl )—N]—, phenyl-(C═O)—NH—, phenyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, phenyl-HN—(C═O)—NH—, (phenyl-)₂N—(C═O)—NH—, phenyl-HN—(C═O)—[((C₁-C₆)alkyl)-N]—, (phenyl-)₂N—(C═O)—[((C₁-C₆)alkyl)-N]—, (C₁-C₆)alkyl-O—(C═O)—NH—, (C₁-C₆)alkyl-O—(C═O)—[((C₁-C₆)alkyl)-N]—, phenyl-O—(C═O)—NH—, phenyl-O—(C═O)—[((C₁-C₆)alkyl)-N]—, (C₁-C₆)alkyl-SO₂NH—, phenyl-SO₂NH—, (C₁-C₆)alkyl-SO₂—, phenyl-SO₂—, hydroxy, (C₁-C₆)alkoxy, perhalo(C₁-C₆)alkoxy, phenoxy, (C₁-C₆)alkyl-(C═O)—O—, phenyl-(C═O)—O—, H₂N—(C═O)—O—, (C₁-C₆)alkyl-HN—(C═O)—O—, [(C₁-C₆)alkyl-]₂N—(C═O)—O—, phenyl-HN—(C═O)—O—, (phenyl-)₂N—(C═O)—O—; wherein each of said moieties containing a phenyl alternative may optionally be substituted by one or two radicals independently selected from the group consisting of (C₁-C₈)alkyl, halo, (C₁-C₆)alkoxy, perhalo(C₁-C₆)alkyl and perhalo(C₁-C₆)alkoxy; more preferably said substituents are independently selected from the group consisting of halo, (C₁-C₆)alkyl, (C₂-C₆)alkenyl, perhalo(C₁-C₆)alkyl, —CN, (C₁-C₆)alkyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, hydroxy, (C₁-C₆)alkoxy, perhalo(C₁-C₆)alkoxy, (C₁-C₆)alkyl-(C═O)—O—, H₂N—(C═O)—O—, (C₁-C₆)alkyl-HN—(C═O)—O— and [(C₁-C₆)alkyl-]₂N—(C═O)—O—.

Another embodiment of the present invention refers to those methods wherein R¹ is optionally substituted (C₁-C₁₀)heteroaryl; wherein said substituents are independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, —NO₂, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, ((C₁-C₆)alkyl-]₂N—(C═O)—[((C₁-C₆)alkyl)-N]—, phenyl-HN—(C═O)—NH—, (phenyl-)₂N—(C═O)—NH—, phenyl-HN—(C═O)—[((C₁-C₆)alkyl)-N]—, (phenyl-)₂N—(C═O)—[((C₁-C₆)alkyl)-N]—, (C₁-C₆)alkyl-O—(C═O)—NH—, (C₁-C₆)alkyl-O—(C═O)—[((C₁-C₆)alkyl)-N]—, phenyl-O—(C═O)—NH—, phenyl-O—(C═O)—[((C₁-C₆)alkyl)-N]—, (C₁-C₆)alkyl-SO₂NH—, phenyl-SO₂NH—, (C₁-C₆)alkyl-SO₂—, phenyl-SO₂—, hydroxy, (C₁-C₆)alkoxy, perhalo(C₁-C₆)alkoxy, phenoxy, (C₁-C₆)alkyl-(C═O)—O—, phenyl-(C═O)—O—, H₂N—(C═O)—O—, (C₁-C₆)alkyl-HN—(C═O)—O—, [(C₁-C₆)alkyl-]₂N—(C═O)—O—, phenyl-HN—(C═O)—O—, (phenyl-)₂N—(C═O)—O—; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy; more preferably said substituents are independently selected from the group consisting of halo, (C₁-C₆)alkyl, (C₂-C₆)alkenyl, perhalo(C₁-C₆)alkyl, —CN, (C₁-C₆)alkyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, hydroxy, (C₁-C₆)alkoxy, perhalo(C₁-C₆)alkoxy, (C₁-C₆)alkyl-(C═O)—O—, H₂N—(C═O)—O—, (C₁-C₆)alkyl-HN—(C═O)—O— and [(C₁-C₆)alkyl-]₂N—(C═O)—O—.

Another preferred embodiment of the present invention refers to those methods wherein R¹ is optionally substituted phenyl; wherein said substituents are independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, —NO₂, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, phenyl-HN—(C═O)—NH—, (phenyl-)₂N—(C═O)—NH—, phenyl-HN—(C═O)—[((C₁-C₆)alkyl)-N]—, (phenyl-)₂N—(C═O)—[((C₁-C₆)alkyl)-N]—, (C₁-C₆)alkyl-O—(C═O)—NH—, (C₁-C₆)alkyl-O—(C═O)—[((C₁-C₆)alkyl)-N]—, phenyl-O—(C═O)—NH—, phenyl-O—(C═O)—[((C₁-C₆)alkyl)-N]—, (C₁-C₆)alkyl-SO₂NH—, phenyl-SO₂NH—, (C₁-C₆)alkyl-SO₂—, phenyl-SO₂—, hydroxy, (C₁-C₆)alkoxy, perhalo(C₁-C₆)alkoxy, phenoxy, (C₁-C₆)alkyl-(C═O)—O—, phenyl-(C═O)—O—, H₂N—(C═O)—O—, (C₁-C₆)alkyl-HN—(C═O)—O—, [(C₁-C₆)alkyl-]₂N—(C═O)—O—, phenyl-HN—(C═O)—O—, (phenyl-)₂N—(C═O)—O—; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy; more preferably said substituents are independently selected from the group consisting of halo, (C₁-C₆)alkyl, (C₂-C₆)alkenyl, perhalo(C₁-C₆)alkyl, —CN, (C₁-C₆)alkyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, hydroxy, (C₁-C₆)alkoxy, perhalo(C₁-C₆)alkoxy, (C₁-C₆)alkyl-(C═O)—O—, H₂N—(C═O)—O—, (C₁-C₆)alkyl-HN—(C═O)—O—and [(C₁-C₆)alkyl-]₂N—(C═O)—O—.

Another embodiment of the present invention refers to those methods wherein R¹ is (R²)₂—N— wherein each R² is independently selected from hydrogen, (C₁-C₆)alkyl, phenyl, (C₁-C₁₀)heterocyclic and (C₃-C₁₀)cycloalkyl; wherein each of the aforesaid R² (C₁-C₆)alkyl, phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O—and phenyl-(C═O)—O—; wherein two R² (C₁-C₆)alkyl groups may be taken together with the nitrogen atom to form a five to six membered heterocyclic or heteroaryl ring.

A more preferred embodiment of the present invention refers to those methods wherein R¹ is (R²)₂—N— wherein each R² is independently selected from hydrogen, (C₁-C₄)alkyl, phenyl and (C₁-C₁₀)heterocyclic; wherein said (C₁-C₄)alkyl, phenyl and (C₁-C₁₀)heterocyclic may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, hydroxy, (C₁-C₆)alkoxy, perhalo(C₁-C₆)alkoxy, amino, (C₁-C₆)alkylamino, [(C₁-C₆)alkyl]₂-amino, (C₁-C₆)alkyl-SO₂—NH—, (C₁-C₆)alkyl-(C═O)—NH—, —CN, (C₁-C₆)alkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)— and (C₁-C₆)alkyl-(C═O)—O—; more preferably optionally substituted with 1-3 substituents independently selected from halo, methyl, hydroxy and amino.

Another embodiment of the present invention refers to those methods wherein R⁴ is hydrogen. Other embodiments of the present invention include those methods wherein R⁴ is hydrogen, in combination with each of the aforementioned R¹ embodiments.

Another embodiment of the present invention refers to those methods wherein R⁴ is R⁵—B—(CH₂)ₙ— and n is zero. Other embodiments of the present invention include those methods wherein R⁴ is R⁵—B—(CH₂)ₙ— and n is zero, in combination with each of the aforementioned R¹ embodiments.

Another embodiment of the present invention refers to those methods wherein R⁴ is R⁵—B—(CH₂)ₙ— and n is an integer from one to six, more preferably one to five, more preferably one to three. Other embodiments of the present invention include those methods wherein R⁴ is R⁵—B—(CH₂)ₙ— and n is an integer from one to five, in combination with each of the aforementioned R¹ embodiments.

Another preferred embodiment of the present invention refers to those methods wherein R⁴ is R⁵—B—(CH₂)ₙ—; n is zero; B is a bond and R⁵ is selected from the group consisting of hydrogen, —CF₃, —C≡—N, (C₁-C₁₀)heteroaryl, (C₁-C₁₀)heterocyclic or (C₃-C₁₀)cycloalkyl; wherein each of the aforesaid (C₁-C₁₀)heteroaryl, (C₁-C₁₀)heterocyclic and (C₃-C₁₀)cycloalkyl may optionally be substituted by one to three moieties independently selected from the group consisting of halo, (C₁-C₆)alkyl, (C₂-C₆)alkenyl, (C₁-C₆)alkynyl, perhalo(C₁-C₆)alkyl, hydroxy, (C₁-C₆)alkoxy, perhalo(C₁-C₆)alkoxy, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, —CN, (C₁-C₆)alkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)— and (C₁-C₆)alkyl-(C═O)—O—. Other embodiments of the present invention include those methods wherein R⁴ is R⁵—B—(CH₂)ₙ— and n is zero; B is a bond and R⁵ is as defined above, in combination with each of the aforementioned R¹ embodiments.

Another embodiment of the present invention refers to those methods wherein R⁴ is R⁵—B—(CH₂)ₙ—; n is zero; B is —(C═O)—NR⁶—, —(R⁶—N)—, —(R⁶—N)—SO₂, —(R⁶—N)—(C═O)—, >C═O, —O—(C═O)—, —SO₂—(NR⁶)—, —(R⁶—N)—(C═O)—(NR⁷)—; and

R⁵ is selected from the group consisting of hydrogen, (C₃-C₁₀)cycloalkyl or phenyl; wherein the aforesaid phenyl and (C₃-C₁₀)cycloalkyl may optionally be substituted by one to three moieties independently selected from the group consisting of halo, (C₁-C₆)alkyl, (C₂-C₆)alkenyl, (C₂-C₆)alkynyl, perhalo(C₁-C₆)alkyl, hydroxy, (C₁-C₆)alkoxy, perhalo(C₁-C₆)alkoxy, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₈)alkyl-(C═O)—NH—, (C₁-C₆)alkyl-(C═O)—[N(C₁-C₆)alkyl]-, —CN, (C₁-C₆)alkyl-(C═O)—, HO—(C═O)—, (C₁-C₆)alkyl-O—(C═O)—, H₂N(C═O)—(C₁-C₆)alkyl-NH—(C═O)—, [(C₁ -C₆)alkyl]₂-N—(C═O)— and (C₁-C₆)alkyl-(C═O)—O—. Other embodiments of the present invention include those methods wherein R⁴ is R⁵—B—(CH₂)ₙ— and n is zero; B is —(C═O)—NR⁶—, —(R⁶—N)—, —(R⁶—N)—SO₂—, —(R⁶—N)—(C═O)—, >C═O, —O—(C═O)—, —SO₂—(NR⁶)—, —(R⁶—N)—(C═O)—(NR⁷)—; and R⁵ is as defined above, in combination with each of the aforementioned refers to those methods R¹ embodiments.

Another preferred embodiment of the present invention refers to those methods wherein R⁴ is R⁵—B—(CH₂)ₙ—; n is zero; B is —(C═O)—NR⁶—, —(R⁶—N)—, >C═O, —O—(C═O)—, —(R⁶—N)—(C═O)— or —(R⁶—N)—(C═O)—(NR⁷)—; R⁵ is R⁹—(R⁸CH)ₘ—; m is 1-6; R⁶ is hydrogen or methyl; R⁸ is hydrogen or methyl; and R⁹ is selected from the group consisting of hydrogen, (C₁-C₆)alkyl, (C₁-C₆)alkoxy, phenyl, (C₁-C₁₀)heteroaryl, (C₁-C₁₀)heterocyclic, (C₃-C₁₀)cycloalkyl, amino, (C₁-C₆)alkylamino, [(C₁-C₆)alkyl]₂amino, (C₁-C₆)alkyl-SO₂—NH—, phenyl-SO₂—NH—, (C₁-C₆)alkyl-SO₂—[N—(C₁-C₆)alkyl]-, phenyl-SO₂—[N—(C₁-C₆)alkyl]-, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[N(C₁-C₆)alkyl]-, phenyl-(C═O)—NH—, phenyl-(C═O)—[N—(C₁-C₆)alkyl]-, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[N—((C₁-C₆)alkyl)]—(C═O)—, (C₁-C₆)alkyl-(C═O)—O— and phenyl-(C═O)—O—. Other embodiments of the present invention include those methods wherein R⁴ is R⁵—B—(CH₂)ₙ— and n is zero; B is —(C═O)—NR⁶—, —(R⁶—N)—, —(R⁶—N)—SO₂—, —(R⁶—N)—(C═O)—, >C═O, —O—(C═O)—, —SO₂—(NR⁶)—, —(R⁶—N)—(C═O)—(NR⁷)—; and R⁵ is R⁹—(R⁸CH)ₘ—; m is 1-6; R⁶ is hydrogen or methyl; R⁸ is hydrogen or methyl; and R⁹ is as defined above, in combination with each of the aforementioned R¹ embodiments.

Another preferred embodiment of the present invention refers to those methods wherein R⁴ is R⁵—B—(CH₂)ₙ—; n is zero; B is —(R⁶—N)—; R⁵ is hydrogen or R⁹—(R⁸CH)ₘ—; m is 1-6; R⁶ is hydrogen or methyl; R⁸ is hydrogen or methyl; and R⁹ is selected from the group consisting of hydrogen, (C₁-C₆)alkyl, hydroxy, (C₁-C₆)alkoxy, amino, (C₁-C₆)alkylamino, [(C₁-C₆)alkyl]₂amino, (C₂-C₆)alkenyl, (C₂-C₆)alkynyl, phenyl, (C₁-C₁₀)heteroaryl, (C₁-C₁₀)heterocyclic and (C₃-C₁₀)cycloalkyl. Other embodiments of the present invention include those methods wherein R⁴ is R⁵—B—(CH₂)ₙ— and n is zero; B is —(R⁶—N)—; R⁵ is hydrogen or R⁹—(R⁸CH)ₘ—; m is 1-6; R⁶ is hydrogen or methyl; R⁸ is hydrogen or methyl; and R⁹ is as defined above, in combination with each of the aforementioned R¹ embodiments.

Another embodiment of the present invention refers to those methods wherein R⁴ is R⁵—B—(CH₂)ₙ—; n is one to six, preferably one to four; B is —(C═O)—NR⁶—, —(R⁶—N)—, —(R⁶—N)—(C═O)— or —(R¹⁰—N)—(C═O)—(NR¹¹)—; R⁹ is R¹³—(R¹²CH)ₘ—; m is 1-6; R¹⁰ is hydrogen or methyl; R⁸ is hydrogen or methyl; and R⁹ is selected from the group consisting of hydrogen, (C₁-C₆)alkyl, (C₁-C₆)alkoxy, phenyl, (C₁-C₁₀)heteroaryl, (C₁-C₁₀)heterocyclic, (C₃-C₁₀)cycloalkyl, amino, (C₁-C₆)alkylamino, [(C₁-C₆)alkyl]₂amino, (C₁-C₆)alkyl-SO₂—NH—, phenyl-SO₂—NH—, (C₁-C₆)alkyl-SO₂—[N—(C₁-C₆)alkyl]-, phenyl-SO₂—[N—(C₁-C₆)alkyl]-, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₆)alkyl-(C═O)—O— and phenyl-(C═O)—O—. Other embodiments of the present invention include those methods wherein R⁴ is R⁵—B—(CH₂)ₙ—; n is one to four; B is —(C═O)—NR⁶—, —(R⁶—N)—, —(R⁶—N)—(C═O)— or —(R⁶—N)—(C═O)—(NR⁷)—; R⁵ is R⁹—(R⁸CH)ₘ—; m is 1-6; R⁶ is hydrogen or methyl; R⁸ is hydrogen or methyl; and R⁹ is as defined above, in combination with each of the aforementioned R¹ embodiments.

Another embodiment of the present invention refers to those methods wherein R⁴ is R ⁵—B—(CH₂)ₙ—; n is an integer from one to six, more preferably one to five, more preferably one to three; B is a bond, and R⁵ is selected from the group consisting of optionally substituted phenyl, (C₁-C₁₀)heterocyclic, (C₁-C₁₀)heteroaryl and (C₃-C₁₀)cycloalkyl; wherein each of the aforesaid R⁵ phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O—and phenyl-(C═O)—O—. Other embodiments of the present invention include those methods wherein R⁴ is R⁵—B—(CH₂)ₙ—; n is an integer from one to six, more preferably one to five, more preferably one to three; B is a bond, and R⁵ is as described above, in combination with each of the aforementioned R¹ embodiments.

Another embodiment of the present invention refers to those methods wherein R⁴ is R⁵—B—(CH₂)ₙ—; n is an integer from one to six, more preferably one to five, more preferably one to three; B is —(C═O)—(R⁶—N)—, —(R⁶—N)—, —SO₂—(R⁶—N)—, —(R⁶—N)—(C═O)—(NR⁷)— or —(R⁶—N)—(C═O)—O—; and R⁵ is selected from the group consisting of optionally substituted phenyl, (C₁-C₁₀)heterocyclic, (C₁-C₁₀)heteroaryl and (C₃-C₁₀)cycloalkyl; wherein each of the aforesaid R⁵ phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—. Other embodiments of the present invention include those methods wherein R⁴ is R⁵—B—(CH₂)ₙ—; n is an integer from one to six, more preferably one to five, more preferably one to three; B is —(C═O)—(R⁶—N)—, —(R⁶—N)—, —SO₂—(R⁶—N)—, —(R⁶—N)—(C═O)—(NR⁷)— or —(R⁶—N)—(C═O)—O—; and R⁵ is as described above, in combination with each of the aforementioned R¹ embodiments.

Another embodiment of the present invention refers to those methods wherein R⁴ is R⁵—B—(CH₂)ₙ—; n is an integer from one to six, more preferably one to five, more preferably one to three; B is a bond, and R⁵ is R⁹—(R° CH)ₘ—; m is 1-6; R⁶ is hydrogen or methyl; each R⁸ is independently selected from the groups consisting of hydrogen or methyl; and R⁹ is selected from the group consisting of hydrogen, (C₁-C₆)alkyl, (C₁-C₆)alkoxy, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, (C₁-C₆)alkyl-SO₂—NH—, phenyl-SO₂—NH—, (C₁-C₆)alkyl-SO₂-[((C₁-C₆)alkyl)-N]—, phenyl-SO₂-[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₆)alkyl-(C═O)—O— and phenyl-(C═O)—O—. Other embodiments of the present invention include those methods wherein R⁴ is R⁵—B—(CH₂)ₙ—; n is an integer from one to six, more preferably one to five, more preferably one to three; B is —(C═O)—(R⁶—N)—, —(R⁶—N)—, —SO₂—(R⁶—N)—, —(R⁶—N)—(C═O)—(NR⁷)— or —(R⁶—N)—(C═O)—O—; R⁵ is R⁹—(R⁸CH)ₘ—; m is 1-6; R⁶ is hydrogen or methyl; each R⁸ is independently selected from the groups consisting of hydrogen or methyl; and R⁹ is as described above, in combination with each of the aforementioned R¹ embodiments.

Another embodiment of the present invention refers to those methods wherein s is an integer from zero to four and each R³ is independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)— and (C₁-C₆)alkyl-(C═O)—O—. Other embodiments of the present invention include those methods wherein R³is as defined above in combination with each of the aforementioned R⁴ embodiments, and/ or with each of the aforementioned R¹ embodiments.

Another embodiment of the present invention refers to those methods wherein s is an integer from zero to four and each R³ is independently selected from the group consisting of halo, —CN, (C₁-C₆)alkyl, (C₂-C₆)alkenyl, (C₂-C₆)alkynyl and perhalo(C₁-C₆)alkyl. Other embodiments of the present invention include those methods wherein R³ is as defined above in combination with each of the aforementioned R⁴ embodiments and/or R¹ embodiments.

Another embodiment of the present invention refers to those methods wherein s is an integer from zero to four and zero, one or two of R³ are independently selected from the group consisting of halo, (C₁-C₆)alkyl, perhalo(C₁-C₆)alkyl, hydroxy, (C₁-C₆)alkoxy, perhalo(C₁-C₆)alkoxy, amino, (C₁-C₆)alkylamino, [(C₁-C₆)alkyl]₂-amino, —CN, and H₂N(C═O)—. Other embodiments of the present invention include those methods wherein R³is as defined above in combination with each of the aforementioned R⁴ embodiments and/or R¹embodiments.

Another embodiment of the present invention refers to those methods wherein s is an integer from zero to four and one of R³ is selected from the group consisting of phenyl, (C₁-C₁₀)heteroaryl, (C₁-C₁₀)heterocyclic and (C₃-C₁₀)cycloalkyl, Other embodiments of the present invention include those methods wherein R³ is as defined above with each of the aforementioned R⁴ embodiments and/or with each of the aforementioned R¹ embodiments.

Another embodiment of the present invention refers to those methods wherein s is an integer from zero to four and one of R³ is selected from the group consisting of hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂— and (C₁-C₆)alkyl-NH—SO₂—. Other embodiments of the present invention include those methods wherein R³ is as defined above in combination with each of the aforementioned R⁴ embodiments and/or with each of the aforementioned R¹ embodiments.

Another embodiment of the present invention refers to those methods wherein s is an integer from zero to four and one of R³ is selected from the group consisting of amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH— and phenyl-(C═O)—[N—(C₁-C₆)alkyl]-. Other embodiments of the present invention include those methods wherein R³ is as defined above in combination with each of the aforementioned R⁴ embodiments and/or with each of the aforementioned R¹ embodiments.

Another embodiment of the present invention refers to those methods wherein s is an integer from zero to four and one of R³ is selected from the group consisting of (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)— and (C₁-C₆)alkyl-(C═O)—O—. Other embodiments of the present invention include those methods wherein R³ is as defined above in combination with each of the aforementioned R⁴ embodiments and/or with each of the aforementioned R¹ embodiments.

Another embodiment of the present invention refers to those methods wherein s is an integer from zero to three and each R³ is independently selected from the group consisting of halo, (C₁-C₆)alkyl, perhalo(C₁-C₆)alkyl, hydroxy, (C₁-C₆)alkoxy, perhalo(C₁-C₆)alkoxy, —NO₂, amino, (C₁-C₆)alkylamino, [(C₁-C₆)alkyl]₂-amino, —CN, and H₂N(C═O)—. Other embodiments of the present invention include those methods wherein R³is as defined above in combination with each of the aforementioned R⁴ embodiments and/or with each of the aforementioned R¹ embodiments.

Another embodiment of the present invention refers to those methods wherein s is an integer from zero to two and each R³ is independently selected from the group consisting of halo, (C₁-C₆)alkyl, perhalo(C₁-C₆)alkyl, (C₁-C₆)alkoxy, perhalo(C₁-C₆)alkoxy and —CN. Other embodiments of the present invention include those methods wherein R³ is as defined above in combination with each of the aforementioned R⁴ embodiments and/or with each of the aforementioned R¹ embodiments.

Another embodiment of the present invention refers to those methods wherein s is an integer from zero to three and each R³ is independently selected from the group consisting of fluoro, chloro and methyl. Other embodiments of the present invention include those methods wherein R³ is as defined above in combination with each of the aforementioned R⁴ embodiments and/or with each of the aforementioned R¹ embodiments.

The present invention also relates to a compound of the formula

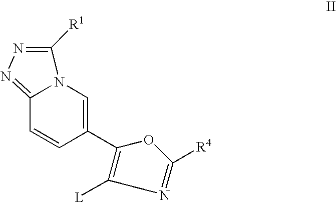

wherein L is bromo, iodo or chloro;

R¹ is selected from the group consisting of hydrogen, —C≡N, (C₁-C₆)alkyl, (C₂-C₆)alkenyl, (C₂-C₆)alkynyl, (C₃-C₁₀)cycloalkyl, phenyl, (C₁-C₁₀)heteroaryl, (C₁-C₁₀)heterocyclic and (R²)₂—N—; wherein each of the aforesaid (C₁-C₆)alkyl, (C₃-C₁₀)cycloalkyl, phenyl, (C₁-C₁₀)heteroaryl and (C₁-C₁₀)heterocyclic substituents may optionally be independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, —NO₂, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, phenyl-HN—(C═O)—NH—, (phenyl-)₂N—(C═O)—NH—, phenyl-HN—(C═O)—[((C₁-C₆)alkyl)-N]—, (phenyl-)₂N—(C═O)—[((C₁-C₆)alkyl)-N]—, (C₁-C₆)alkyl-O—(C═O)—NH—, (C₁-C₆)alkyl-O—(C═O)—[((C₁-C₆)alkyl)-N]—, phenyl-O—(C═O)—NH—, phenyl-O—(C═O)—[((C₁-C₆)alkyl)-N]—, (C₁-C₆)alkyl-SO₂NH—, phenyl-SO₂NH—, (C₁-C₆)alkyl-SO₂—, phenyl-SO₂—, hydroxy, (C₁-C₆)alkoxy, perhalo(C₁-C₆)alkoxy, phenoxy, (C₁-C₆)alkyl-(C═O)—O—, phenyl-(C═O)—O—, H₂N—(C═O)—O—, (C₁-C₆)alkyl-HN—(C═O)—O—, [(C₁-C₆)alkyl-]₂N—(C═O)—O—, phenyl-HN—(C═O)—O—, (phenyl-)₂N—(C═O)—O—; wherein when said R¹ phenyl contains two adjacent substituents, such substituents may optionally be taken together with the carbon atoms to which they are attached to form a five to six membered carbocyclic or heterocyclic ring; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy;

each R² is independently selected from hydrogen, (C₁-C₆)alkyl, phenyl, (C₁-C₁₀)heteroaryl, (C₁-C₁₀)heterocyclic and (C₃-C₁₀)cycloalkyl; wherein each of the aforesaid R² (C₁-C₆)alkyl, phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C_{1-C}₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O—and phenyl-(C═O)—O—; wherein two R² (C₁-C₆)alkyl groups may be taken together with the nitrogen atom to which they are attached to form a five to six membered heterocyclic or heteroaryl ring;

R⁴ is selected from the group consisting of hydrogen, halo or R⁵—B—(CH₂)ₙ—;

n is an integer from zero to six;

each B is independently a bond, —(CHR⁶)—, —O—, —S—, —(SO₂)—, —(C═O)—, —O—(C═O)—, —(C═O)—O—, —(C═O)—NR⁶—, —(R⁶—N)—, —(R⁶—N)—SO₂—, —(R⁶—N)—(C═O)—, —SO₂—(NR⁶)—, —(R¹⁰—N)—(C═O)—(NR⁷)—, —(O)—(C═O)—(NR⁶)— or —(R⁶—N)—(C═O)—O—;

R⁵ is selected from the group consisting of hydrogen, —CF₃, —C≡—N, R⁹—(R⁸CH)ₘ—, phenyl, (C₁-C₁₀)heterocyclic, (C₁-C₁₀)heteroaryl, and (C₃-C₁₀)cycloalkyl; wherein each of the aforesaid R⁵ phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl)₂amino, (C₁-C₆)alkyl-SO₂—NH—, (C₁-C₆)alkyl-(C═O)—NH—, (C₁-C₆)alkyl-(C═O )—(((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O—and phenyl-(C═O)—O—; wherein two adjacent R⁵ substituents of said phenyl, (C₁-C₁₀)heteroaryl, (C₁-C₁₀)heterocyclic and (C₃-C₁₀)cycloalkyl may optionally be taken together with the carbon or heteroatom to which they are attached to form a five or six membered carbocyclic or heterocyclic ring;

m is an integer from one to six;

R⁶ is hydrogen, (C₁-C₆)alkyl-SO₂— or (C₁-C₆)alkyl;

R⁷ is hydrogen or (C₁-C₆)alkyl;

each R⁸ is independently selected from the group consisting of hydrogen, amino, (C₁-C₆)alkoxy and (C₁-C₆)alkyl;

R⁹ is selected from the group consisting of hydrogen, (C₁-C₆)alkyl, (C₂-C₆)alkenyl, (C₂-C₆)alkynyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, phenyl-SO₂—NH—, (C₁-C₆)alkyl-SO₂—[((C₁-C₆)alkyl)-N]—, phenyl-SO₂—[((C₁-C₆)alkyl)-N]—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O—and phenyl-(C═O)—O—;

or a salt thereof.

The present invention also relates to a compound of the formula

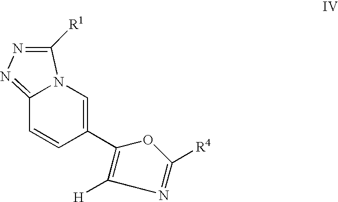

wherein R¹ and R⁴ are as defined above in claim 21; or a salt thereof.

The present invention also relates to a compound of the formula

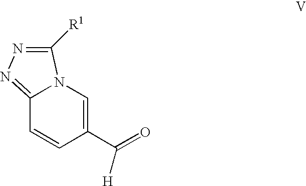

wherein R¹ is as defined above; or a salt thereof.

The present invention also relates to the acceptable acid addition salts of compounds of the formulae I, II, IV, and V. The acids which are used to prepare the acceptable acid addition salts of the aforementioned base compounds of this invention are those which form non-toxic acid addition salts, i.e., salts containing pharmacologically acceptable anions, such as the chloride, bromide, iodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3-naphthoate)]salts.

The invention also relates to base addition salts of formulae I, II, IV and V. The chemical bases that may be used as reagents to prepare acceptable base salts of those compounds of formula I that are acidic in nature are those that form non-toxic base salts with such compounds. Such non-toxic base salts include, but are not limited to those derived from such acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of acceptable organic amines.

The compounds of this invention include all stereoisomers (e.g., cis and trans isomers) and all optical isomers of compounds of the formula I (e.g., R and S enantiomers), as well as racemic, diastereomeric and other mixtures of such isomers.

The compounds and prodrugs of the present invention can exist in several tautomeric forms, including the enol and imine form, the keto and enamine form and geometric isomers and mixtures thereof. All such tautomeric forms are included within the scope of the present invention. Tautomers exist as mixtures of tautomers in solution. In solid form, usually one tautomer predominates. Even though one tautomer may be described, the present invention includes all tautomers of the present compounds.

The present invention also includes atropisomers of the present invention. Atropisomers refer to compounds of formula I that can be separated into rotationally restricted isomers.

The compounds of this invention may contain olefin-like double bonds. When such bonds are present, the compounds of the invention exist as cis and trans configurations and as mixtures thereof.

A “suitable substituent” is intended to mean a chemically and pharmaceutically acceptable functional group i.e., a moiety that does not negate the inhibitory activity of the inventive compounds. Such suitable substituents may be routinely selected by those skilled in the art. Illustrative examples of suitable substituents include, but are not limited to halo groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, amino groups, alkyl- and dialkylamino groups, carbamoyl groups, alkylcarbonyl groups, alkoxycarbonyl groups, alkylaminocarbonyl groups dialkylamino carbonyl groups, arylcarbonyl groups, aryloxycarbonyl groups, alkylsulfonyl groups, arylsulfonyl groups and the like.

As used herein, the term “alkyl,” as well as the alkyl moieties of other groups referred to herein (e.g., alkoxy), may be linear or branched (such as methyl, ethyl, n-propyl, isopropyl, n-butyl, iso-butyl, secondary-butyl, tertiary-butyl), and they may also be cyclic (e.g., cyclopropyl or cyclobutyl); optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. The phrase “each of said alkyl” as used herein refers to any of the preceding alkyl moieties within a group such alkoxy, alkenyl or alkylamino. Preferred alkyls include (C₁-C₄)alkyl, most preferably methyl.

As used herein, the term “cycloalkyl” refers to a mono or bicyclic carbocyclic ring (e.g., cyclopropyl, cyclobutyl, cyclopentyl, cyclohexyl, cycloheptyl, cyclooctyl, cyclononyl, cyclopentenyl, cyclohexenyl, bicyclo[2.2.1]heptanyl, bicyclo[3.2.1]octanyl and bicyclo[5.2.0]nonanyl, etc.); optionally containing 1-2 double bonds and optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. The phrase “each of said alkyl” as used herein refers to any of the preceding alkyl moieties within a group such alkoxy, alkenyl or alkylamino. Preferred cycloalkyls include cyclobutyl, cyclopentyl and cyclohexyl.

As used herein, the term “halogen” includes fluoro, chloro, bromo or iodo or fluoride, chloride, bromide or iodide.

As used herein, the term “halo-substituted alkyl” refers to an alkyl radical as described above substituted with one or more halogens included, but not limited to, chloromethyl, dichloromethyl, fluoromethyl, difluoromethyl, trifluoromethyl, 2,2,2-trichloroethyl, and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “alkenyl” means straight or branched chain unsaturated radicals of 2 to 6 carbon atoms, including, but not limited to ethenyl, 1-propenyl, 2-propenyl (alkyl), iso-propenyl, 2-methyl-1-propenyl, 1-butenyl, 2-butenyl, and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “(C₂-C₆)alkynyl” is used herein to mean straight or branched hydrocarbon chain radicals having one triple bond including, but not limited to, ethynyl, propynyl, butynyl, and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “carbonyl” or “(C═O)” (as used in phrases such as alkylcarbonyl, alkyl-(C═O)— or alkoxycarbonyl) refers to the joinder of the >C═O moiety to a second moiety such as an alkyl or amino group (i.e. an amido group). Alkoxycarbonylamino (i.e. alkoxy(C═O)—NH—) refers to an alkyl carbamate group. The carbonyl group is also equivalently defined herein as (C═O). Alkylcarbonylamino refers to groups such as acetamide.

As used herein, the term “phenyl-[((C₁-C₆)alkyl)-N]—(C═O)—, ” as used herein, refers to a disubstituted amide group of the formula

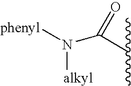

As used herein, the term “aryl” means aromatic radicals such as phenyl, naphthyl, tetrahydronaphthyl, indanyl and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “heteroaryl” refers to an aromatic heterocyclic group usually with one heteroatom selected from O, S and N in the ring. In addition to said heteroatom, the aromatic group may optionally have up to four N atoms in the ring. For example, heteroaryl group includes pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. Particularly preferred heteroaryl groups include oxazolyl, imidazolyl, pyridyl, thienyl, furyl, thiazolyl and pyrazolyl (these heteroaryls are most preferred of the R⁴ heteroaryls).

The term “heterocyclic” as used herein refers to a cyclic group containing 1-9 carbon atoms and 1-4 hetero atoms selected from N, O, S or NR′. Examples of such rings include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydrothiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl and the like. Examples of such monocyclic saturated or partially saturated ring systems are tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, 1,4-oxazin-2-yl, 1,2,5-oxathiazin-4-yl and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. Preferred heterocyclics include tetrahydrofuranyl, pyrrolidinyl, piperidinyl, piperazinyl and morpholinyl.

More specifically, the present invention also relates to a compound of the formulae I, II, IV and V wherein R¹ is (C₁-C₆)alkyl, more preferably wherein R¹ is isopropyl.

Another embodiment of the present invention relates to a compound of the formulae I, II, IV and V wherein R⁴is hydrogen.

Another embodiment of the present invention relates to a compound of the formulae I, II, IV and V, wherein R⁴ is R⁵—B—(CH₂)ₙ— and n is zero.

Another embodiment of the present invention relates to a compound of the formulae I, II, IV and V wherein R⁴is R⁵—B—(CH₂)ₙ— and n is an integer from one to five.

Another embodiment of the present invention relates to a compound of the formulae I, II, IV and V, wherein R⁴ is R⁵—B—(CH₂)ₙ—; n is zero; B is a bond and R⁵ is selected from the group consisting of hydrogen, —CF₃, —C≡N, (C₁-C₁₀)heteroaryl, (C₁-C₁₀)heterocyclic or (C₃C₁₀)cycloalkyl; wherein each of the aforesaid (C₁-C₁₀)heteroaryl, (C₁-C₁₀)heterocyclic and (C₃C₁₀)cycloalkyl may optionally be substituted by one to three moieties independently selected from the group consisting of halo, (C₁-C₆)alkyl, (C₂-C₆)alkenyl, (C₁-C₆)alkynyl, perhalo(C₁-C₆)alkyl, hydroxy, (C₁-C₆)alkoxy, perhalo(C₁-C₆)alkoxy, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂NH—, (C₁-C₆)alkyl-(C═O)—NH—, (C₁-C₆)alkyl-(C═O)—[((C₁-C₆)alkyl)-N]—, —CN, (C₁-C₆)alkyl(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)— and (C₁-C₆)alkyl-(C═O)—O—.

Another embodiment of the present invention relates to a compound of the formulae I, II, IV and V, wherein R⁴ is R⁵—B—(CH₂)ₙ—; n is zero; B is —(C═O)—NR⁶—, —(R⁶—N)—, —(R⁶—N)—SO₂—, (R⁶—N)—(C═O)—,>C═O, —O—(C═O)—, —SO₂—(NR⁶)—, —(R⁶—N)—(C═O)—(NR⁶)—; and

R⁵ is selected from the group consisting of hydrogen, (C₃-C₁₀)cycloalkyl or phenyl; wherein the aforesaid phenyl and (C₃-C₁₀)cycloalkyl may optionally be substituted by one to three moieties independently selected from the group consisting of halo, (C₁-C₆)alkyl, (C₂-C₆)alkenyl, (C₂-C₆)alkynyl, perhalo(C₁-C₆)alkyl, hydroxy, (C₁-C₆)alkoxy, perhalo(C₁-C₆)alkoxy, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[N(C₁-C₆)alkyl]-, —CN, (C₁-C₆)alkyl-(C═O)—, HO—(C═O)—, (C₁-C₆)alkyl—O—(C═O)—, H₂N(C═O)—(C₁-C₆)alkyl-NH—(C═O)—, [(C₁-C₆)alkyl]₂-N—(C═O)— and (C₁-C₆)alkyl-(C═O)—O—.

Another embodiment of the present invention relates to a compound of the formulae I, II, IV and V, wherein R⁴ is R⁵—B—(CH₂)ₙ—; n is zero; B is —(C═O)—NR—, —(R⁶—N)—,>C═O, —O—(C═O)—, —(R⁶—N)—(C═O)— or —(R⁶—N)—(C═O)—(NR⁷)—; R⁵ is R⁹—(R⁸CH)ₘ—; m is 1-6; R⁶ is hydrogen or methyl; R⁸ is hydrogen or methyl; and R⁹ is selected from the group consisting of hydrogen, (C₁-C₆)alkyl, (C₁-C₆)alkoxy, phenyl, (C₁-C₁₀)heteroaryl, (C₁-C₁₀)heterocyclic, (C₃-C₁₀)cycloalkyl, amino, (C₁-C₆)alkylamino, [(C₁-C₆)alkyl]₂amino, (C₁-C₆)alkyl-SO₂—NH—, phenyl-SO₂—NH—, (C₁-C₆)alkyl-SO₂—[N—(C₁-C₆)alkyl]-, phenyl-SO₂—[N—(C₁-C₆)alkyl]-, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl)]₂amino, (C₁-C₆)alkyl-SO₂—NH—, (C₁-C₆)alkyl-(C═O)—NH—, (C₁-C₆)alkyl-(C═O)—[N (C₁-C₆)alkyl]-, phenyl-(C═O)—NH—, phenyl-(C═O)—[N—(C₁-C₆)alkyl]-, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[N—((C₁-C₆)alkyl)]-(C═O)—, (C₁-C₆)alkyl-(C═O)—O— and phenyl-(C═O)—O—.

Another embodiment of the present invention relates to a compound of the formulae I, II, IV and V, wherein R⁴ is R⁵—B—(CH₂)ₙ—; n is zero; B is —(R⁶—N)—; R⁵ is hydrogen or R⁹(R⁸CH)ₘ—; m is 1-6; R⁶ is hydrogen or methyl; R⁸ is hydrogen or methyl; and R⁹ is selected from the group consisting of hydrogen, (C₁-C₆)alkyl, hydroxy, (C₁-C₆)alkoxy, amino, (C₁-C₆)alkylamino, [(C₁-C₆)alkyl]₂amino, (C₂-C₆)alkenyl, (C₂-C₆)alkynyl, phenyl, (C₁-C₁₀)heteroaryl, (C₁-C₁₀)heterocyclic and (C₃-C₁₀)cycloalkyl.

Another embodiment of the present invention relates to a compound of the formulae I, II, IV and V, wherein R⁴ is R⁵—B—(CH₂)ₙ—; n is one to four; B is —(C═O)—NR⁶—, —(R⁶—N)—, —(R⁶—N)—(C═O)— or —(R⁶—N)—(C═O)—(NR⁷)—; R⁵ is R⁹—(R⁸CH)ₘ—; m is 1-6; R⁶ is hydrogen or methyl; R ⁸is hydrogen or methyl; and R⁹ is selected from the group consisting of hydrogen, (C₁-C₆)alkyl, (C₁-C₆)alkoxy, phenyl, (C₁-C₁₀)heteroaryl, (C₁-C₁₀)heterocyclic, (C₃-C₁₀)cycloalkyl, amino, (C₁-C₆)alkylamino, [(C₁-C₆)alkyl]₂amino, (C₁-C₆)alkyl-SO₂—NH—, phenyl-SO₂—NH—, (C₁-C₆)alkyl-SO₂—[N—(C₁-C₆)alkyl]-, phenyl-SO₂—[N—(C₁-C₆)alkyl]-, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, HO—(C═O)—, (C₁-C₆)alkyl-O—(C═O)—, H₂N(C═O)-(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₆)alkyl-(C═O)—O— and phenyl-(C═O)—O—.

Another embodiment of the present invention relates to a compound of the formulae I, II, IV and V, wherein s is an integer from zero to four and each R³ is independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-, amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)— and (C₁-C₆)alkyl-(C═O)—O—.

Another embodiment of the present invention relates to a compound of the formulae I, II, IV and V, wherein s is an integer from zero to four and each R³ is independently selected from the group consisting of halo, —CN, (C₁-C₆)alkyl, (C₂-C₆)alkenyl, (C₂-C₆)alkynyl and perhalo(C₁-C₆)alkyl.

Another embodiment of the present invention relates to a compound of the formulae I, II, IV and V, wherein s is an integer from zero to four and zero, one or two of R³ are independently selected from the group consisting of halo, (C₁-C₆)alkyl, perhalo(C₁-C₆)alkyl, hydroxy, (C₁-C₆)alkoxy, perhalo(C₁-C₆)alkoxy, amino, (C₁-C₆)alkylamino, [(C₁-C₆)alkyl]₂-amino, —CN, and H₂N(C═O)—.

Another embodiment of the present invention relates to a compound of the formulae I, II, IV and V, wherein s is an integer from zero to three and each R³ is independently selected from the group consisting of halo, (C₁-C₆)alkyl, perhalo(C₁-C₆)alkyl, hydroxy, (C₁-C₆)alkoxy, perhalo(C₁-C₆)alkoxy, —NO₂, amino, (C₁-C₆)alkylamino, [(C₁-C₆)alkyl]₂-amino, —CN, and H₂N(C═O)—.

Another embodiment of the present invention relates to a compound of the formulae I, II, IV and V, wherein s is an integer from zero to two and each R³ is independently selected from the group consisting of halo, (C₁-C₆)alkyl, perhalo(C₁-C₆)alkyl, (C₁-C₆)alkoxy, perhalo(C₁-C₆)alkoxy and —CN.

Another embodiment of the present invention relates to a compound of the formulae I, II, IV and V, wherein s is an integer from zero to three and each R³ is independently selected from the group consisting of fluoro, chloro and methyl.

Specific compounds of the invention consisting of:

3-Isopropyl-6-[4-bromo-oxazol-5-yl]-[1,2,4]triazolo[4,3-a]pyridine; and

3-Isopropyl-6-[oxazol-5-yl]-[1,2,4]triazolo[4,3-a]pyridine;

or pharmaceutically acceptable salts thereof.

The present invention also includes isotopically-labelled compounds, which are identical to those recited in Formula I, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ³C ¹⁴C, ¹⁵N ¹⁷O, ³¹P, ⁵⁰P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

Compounds of Formula (I) are capable of inhibiting proinflammatory cytokines, such as IL-1, IL-6, IL-8, and TNF and are therefore of use in therapy. IL-1, IL-6, IL-8 and TNF affect a wide variety of cells and tissues and these cytokines, as well as other leukocyte-derived cytokines, are important and critical inflammatory mediators of a wide variety of disease states and conditions. The inhibition of these pro-inflammatory cytokines is of benefit in controlling, reducing and alleviating many of these disease states.

### DETAILED DESCRIPTION OF THE INVENTION

Compounds of the formula I may be prepared according to the following reaction schemes and discussion. Unless otherwise indicated, m, n, s, B, R¹ through R⁹ and Het and structural formulae I, II, IV and V in the reaction schemes and discussion that follow are as defined above.

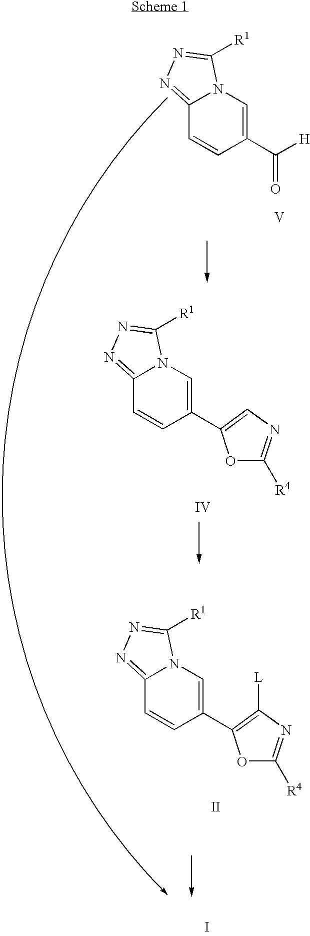

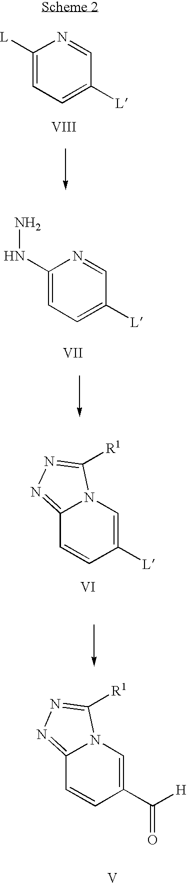

Scheme 1 refers to the preparation of compounds of the formula I. Referring to Scheme 1, compounds of the formula I can be prepared from compounds of the formula II by reaction with a compound of the formula

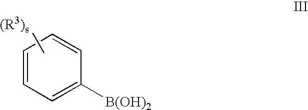

a transition metal catalyst, and a base. Suitable catalysts include palladium (such as palladium acetate (Pd(OAc)₂), tetrakis (triphenylphosphine) palladium (0), Pd(dppf)Cl₂, tris(dibenzylidene acetone)dipalladium(0) (Pd₂(dba)₃), and di(dibenzylidene acetone) palladium(0) (Pd(dba)₂)), preferably tetrakis (triphenylphosphine)palladium(0). Suitable bases include tertiary amine bases, such as triethylamine or pyridine, Na₂CO₃, sodium ethoxide, and K₃PO₄, preferably triethylamine. Suitable solvents include alcohols, such as methanol, ethanol and butanol, methylene chloride, dimethyl sulfoxide (DMSO) or tetrahydrofuran (THF), preferably ethanol. The aforesaid reaction is typically performed under an atmosphere of nitrogen gas at a temperature of about 10° C. to 50° C., preferably about 23° C. (room temperature) for about 6 to 72 hours. Palladium-catalyzed boronic acid couplings are described in Miyaura, N., Yanagi, T., Suzuki, A. Syn. Comm. 1981, 11, 7, p. 513.

The compound of formula II, wherein L is Br, can be prepared from a compound of formula IV by reaction with a suitable bromination reagent such as phenyl trimethylammonium tribromide, N-bromosuccinimide, pyridinium bromide, perbromide, Br₂ or Br₂-Ph₃P, preferably N-bromosuccinimide. The bromination may be carried out in a reaction inert solvent such as N,N-dimethylformamide, diethyl ether or tetrahydrofuran, preferably N,N-dimethylformamide. The aforesaid reaction is conducted at a temperature of about −78° C. to about 40° C. preferably about −78° C. to about 0° C. for a time period between about 1 hour to about 16 hours. Preferably, the reaction is conducted in the presence of a base such as lithium bis(trimethylsilyl)amide.

The compound of formula IV can be prepared from a compound of the formula V by reaction with tosylmethyl isocyanide in the presence of a base in a solvent. Suitable bases include alkali metal carbonates or hydroxide bases, preferably potassium carbonate. Suitable solvents for the aforesaid reaction include hexane, methylene chloride, alcohols, N,N-dimethylformamide (DMF), N,N-dimethylacetamide or N-methylpyrrolidinone (NMP) preferably methanol. The aforesaid reaction may be run at a temperature between about 30° C. and 180° C., preferably about 65° C., for about 30 minutes to 24 hours, preferably about 2 hours.

Alternatively, a compound of the formula I can be prepared from aldehydes of formula V by reaction with an isocyanide of formula

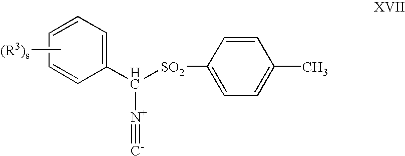

in the presence of a base. Suitable bases include potassium carbonate, triethylamine, and piperazine, preferably potassium carbonate. Suitable solvents include polar solvents such as tetrahydrofuran, acetonitrile or N,N-dimethylformamide, preferably in acetonitrile or THF. The aforesaid reaction may be run at a temperature between about 22° C. and about 70° C., preferably at about 22° C. for a period from about 2 hours to about 4 hours, followed by about 6 hours to about 10 hours at a temperature of about 70° C.

The compound of the formula

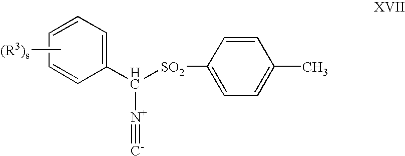

may be prepared by reacting a compound of the formula

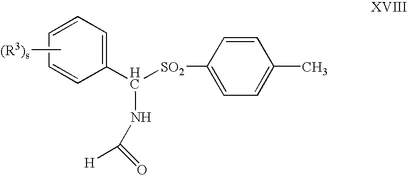

with a dehydrating agent such as POCl₃, and a weak hindered base such as 2,6-lutidine or 2,4,6-trimethyl pyridine. Preferably the reaction is performed in the presence of a solvent such as tetrahydrofuran, dimethyl ether or methylene chloride. The aforesaid reaction may be run at a temperature between about −20° C. and about 50° C., preferably at about 0° C. to about room temperature for a period from about 2 hours to about 48 hours, preferably about 24 hours.

Scheme 2 refers to the preparation of compounds of the formula V which are intermediates useful in the preparation of compounds of the formula I in Scheme I. Referring to Scheme 2, compounds of formula V are prepared from compounds of formula VI by a formylation reaction. Suitable conditions for formylation include reaction with an (C₁-C₆)alkyl magnesium halide or (C₁-C₆)alkyl lithium, followed by reaction with a disubstituted formamide reagent. Preferably the work-up of the aforesaid reaction is done in the absence of a strong acid or base, such as with aqueous citric acid or potassium phosphate. The aforesaid reaction is performed in a solvent such as tetrahydrofuran at a temperature of about −30° C. to about 50° C., for a period of time of about 5 minutes to about 24 hours, followed by the addition of N,N-dimethylformamide at a temperature of about 0° C., followed by a period of time of about 2 hours to about 24 hours at a temperature of about 40° C. to about 100° C.

Compounds of formula VI are prepared as described in the literature (Moran, D. B.; Morton, G. O.; Albright, J. D., J. Heterocycl. Chem., Vol. 23, pp. 1071-1077 (1986)) or from compounds of formula VII, wherein L′ is bromo or iodo, by reaction with a cyclization reagent such as acid anhydride or an acid chloride, more preferably isobutyl chlorate or a reagent of the formula Y—(C═O)_{R} I. Compounds of formula VIII are commercially available.

The compounds of the formulae I, II, IV and V which are basic in nature are capable of forming a wide variety of different salts with various inorganic and organic acids. Although such salts must be pharmaceutically acceptable for administration to animals, it is often desirable in practice to initially isolate a compound of the formula I from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [i.e., 1,1′-methylene-bis-(2-hydroxy-3-naphthoate)] salts.

Those compounds of the formulae I, II, IV and V which are also acidic in nature, e.g., where R¹-R⁹ includes a COOH or tetrazole moiety, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the herein described acidic compounds of formula I. These non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

The activity of the compounds of the invention for the various disorders described above can be determined according to one or more of the following assays. All of the compounds of the invention, that were tested, had an IC₅₀ of less than 10 μM in the TNFα and MAPKAP in vitro assays and an ED₅₀ of less than 50 mg/kg in the in vivo TNFα assay.

The compounds of the present invention also possess differential activity (i.e. are selective for) for one or more p38 kinases (i.e. α, β, γ, and δ). Certain compounds are selective for p38α over p38β, γ, and δ, other compounds are selective for p38β over p38α, γ, and δ, other compounds are selective for p38 α and β over p38 γ and δ. Selectivity is measured in standard assays as a IC₅₀ ratio of inhibition in each assay.

Inhibition of TNF-Alpha Production by Human LPS-Treated Monocytes

Mononuclear cells are isolated from heparinized blood (1.5 ml of 1000 units/ml heparin for injection, Elkins-Sinn, Inc. added to each 50 ml sample) using Accuspin System-Histopaque-1077 tubes (Sigma A-7054). Thirty-five milliliters of whole blood are added of each tube and the tubes are centrifuged at 2100 rpm for 20 minutes in a Beckman GS-6KR centrifuge with the brake off at room temperature. The mononuclear cells which collect at the interface are removed, diluted with Macrophage serum free medium (Gibco-BRL) (Medium) to achieve a final volume of 50 ml, and collected by centrifugation for 10 minutes. The supernatant is discarded and the cell pellet is washed 2 times with 50 ml of Medium. A sample of the suspended cells is taken before the second wash for counting. Based on this count, the washed cells are diluted with Medium containing 1% FBS to a final concentration of 2.7×10⁶ cells/ml and 75 μl of the cell suspension is added to each well of a 96 well plate.

Compound Preparation

Compounds are routinely tested at final concentrations from 2 μM to 0.016 μM, but may be tested at other concentrations, depending on activity. Test agents are diluted with DMSO to a final concentration of 2 mM. From this stock solution, compounds are first diluted 1:25 (5 μl of 2 mM stock+120 μl Medium containing 400 ng/ml LPS and 1% FBS then 40 μl of this dilution is diluted with 360 μl of Medium with LPS. Serial dilutions (1/5) are performed by transferring 20 μl of this dilution to 80 μl of Medium containing both LPS and 0.4% DMSO, resulting in solutions containing 8 μM, 1.6 μM, 0.32 μM and 0.064 μM of test agent.

Assay

The assay is initiated by adding 25 μl of the diluted compounds to the mononuclear cell suspension and incubating the cells at 37 C and 5% CO₂ for 4 hours.

The 96-well plates are then centrifuged for 10 minutes at 2000 rpm at 4° C. in a Beckman GS-6KR centrifuge to remove cells and cell debris. A 90 μl aliquot of each supernatant is removed and transferred to a 96 well round bottom plate, and this plate is centrifuged a second time to insure that all cell debris is removed. 80 μl of the supernatant is removed and transferred to a new round bottom plate.

Supernatants are analyzed for TNF-α content using R&D ELISA. 25 μl of each sample is added to an ELISA well containing 25 μl of assay diluent RD1F and 75 μl of assay diluent RD5. The assay is run following kit directions except 100 μl of conjugate and substrate solutions are used.

### Interpretation

The amount of TNF-α immunoreactivity in the samples is calculated as follows:

% Control=(X−B)/(TOT−B)×100

where X=OD₄₅₀ nm of the test compound well

B=OD₄₅₀ of Reagent Blank wells on the ELISA

Total=OD₄₅₀ of cells that were treated with 0.1% DMSO only.

### MAPKAP Kinase-2 Assay

Monocyte Preparation

Mononuclear cells are collected from heparinized human blood as detailed above. The washed cells are seeded into 6-well cluster plates at a density of 1×10⁷ cells/well (in 2 ml of Medium). The plates are incubated at 37° C. in a 5% CO₂ environment for 2 hours to allow adherence of the monocytes, after which time media supernatants containing non-adherent cells are removed by aspiration and 2 ml of fresh medium are added to each well. Plates are incubated overnight at 37° C. in a 5% CO₂ environment.

Cell Activation

Media are removed by aspiration. The attached cells are rinsed twice with fresh Medium, then 2 ml of D-MEM medium containing 10% heat inactivated FBS are added to each well. Test compounds are prepared as 30 mM stock solutions in DMSO and diluted to 1250, 250, 50, 10, 2, and 0.4 μM in D-MEM containing 1% DMSO and 10% FBS. To individual wells of the monocyte cultures, 20 μl of these test agent dilutions are added resulting in final test agent concentrations of 12.5, 2.5, 0.5, 0.1, 0.02 and 0.004 μM. After a 10 minute preincubation period, 20 μl of a 10 μg/ml LPS solution are added to each well and the plates are incubated at 37° C. for 30 minutes. Media subsequently are removed by aspiration, the attached monocytes are rinsed twice with phosphate buffered saline, then 1 ml of phosphate buffered saline containing 1% Triton X-100 (Lysis Buffer; also containing 1 Complete™ tablet [Boehringer #1697498] per 10 ml of buffer) is added to each well. The plates are incubated on ice for 10 minutes, after which the lysates are harvested and transferred to centrifugation tubes. After all samples are harvested, they are clarified by centrifugation (45,000 rpm for 20 minutes) and the supernatants recovered.

MAPKAP Kinase-2 Immunoprecipitation

5 μl of anti-MAPKAP kinase-2 antiserum (Upstate Biotechnology #06-534) is added to a microcentrifuge tube (1 tube for each of the above cell lysates) containing 1 ml of a 5% suspension of Protein G-Sepharose (Sigma #P3296) in PBS. These mixtures are incubated for 1 hour at 4° C. (with rocking) after which the beads, containing bound IgG, are recovered by centrifugation and washed twice with 1 ml of 50 mM Tris, pH 7.5, 1 mM EDTA, 1 mM EGTA, 0.5 mM orthovanadate, 0.1% 2-mercaptoethanol, 1% Triton X-100, 5 mM sodium pyrophosphate, 10 mM sodium β-glycerophosphate, 0.1 mM phenylmethylsulfonyl fluoride, 1 μg/ml leupeptin, 1 μg/ml pepstatin, and 50 mM sodium fluoride (Buffer A) by repeated centrifugation. An individual monocyte cell extract (prepared above) is then transferred to each tube containing a pellet of IgG-coated Protein G-Sepharose, and these mixtures are incubated for 2 hours at 4° C. (with rocking). The beads subsequently are harvested by centrifugation, and the resulting bead pellets are washed once with 0.5 ml of Buffer A containing 0.5 M NaCl, once with 0.5 ml of Buffer A, and once with 0.1 ml of a buffer composed of 20 mM MOPS, pH 7.2, 25 mM sodium β-glycerophosphate 5 mM EGTA, 1 mM orthovanadate, and 1 mM dithiothreitol (Buffer B).

MAPKAP Kinase-2 Activity Assessment

A kinase reaction mixture stock is prepared as follows: 2.2 μl of 10 mCi/ml γ[³²P]ATP, 88 μl of 1.3 μg/ml solution of MAPKAP Kinase-2 substrate peptide (Upstate Biotechnology #12-240), 11 μl of 10 mM ATP, 8.8 μl of 1 M MgCl₂, and 770 μl of Buffer B. To each of the immune complex-Protein G-pellets, 40 μl of the kinase reaction mixture are added and the tubes are incubated for 30 minutes at 30° C. The tubes then are clarified by centrifugation and 25 μl of each supernatant is spotted onto a P81 filter paper disk (Whatman #3698-023). After allowing all fluid to soak into the filter, each disk is placed into an individual well of 6-well cluster plates and the filters are washed sequentially with 2 ml of 0.75% phosphoric acid (3 washes/15 minutes each) and once with acetone (10 minutes). The filters then are air dried and transferred to liquid scintillation vials containing 5 ml of scintillation fluid. Radioactivity is determined in a liquid scintillation counter. The amount of radioactivity bound to the filter at each test agent concentration is expressed as a percentage of that observed from cells stimulated with LPS in the absence of a test agent.

### In Vivo Inhibition of TNFα

Rats were weighed and dosed with vehicle (0.5% methyl cellulose, Sigma) or drug. One hour later, animals were injected i.p. with LPS (50 ug/rat, Sigma L-4130). Ninety minutes later, animals were sacrificed by asphyxiation with CO₂ and bled by cardiac puncture. Blood was collected in Vaccutainer tubes and spun for 20 minutes at 3000 rpm. Serum was assayed for TNFα levels using an ELISA (R&D Systems).

This invention also encompasses pharmaceutical compositions containing and methods of treating or preventing comprising administering prodrugs of compounds of the formula I. Compounds of formula I having free amino, amido, hydroxy or carboxylic groups can be converted into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of formula I. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds wherein carbonates, carbamates, amides and alkyl esters which are covalently bonded to the above substituents of formula I through the carbonyl carbon prodrug sidechain.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The compounds of formula I can also be formulated for sustained delivery according to methods well known to those of ordinary skill in the art. Examples of such formulations can be found in U.S. Pat. Nos. 3,538,214, 4,060,598, 4,173,626, 3,119,742, and 3,492,397, which are herein incorporated by reference in their entirety.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., inflammation) is 0.1 to 200 mg of the active ingredient per unit dose which could be administered, for example, 1 to 4 times per day.

Aerosol formulations for treatment of the conditions referred to above (e.g., adult respiratory distress syndrome) in the average adult human are preferably arranged so that each metered dose or “puff” of aerosol contains 20 μg to 1000 μg of the compound of the invention. The overall daily dose with an aerosol will be within the range 100 μg to 10 mg. Administration may be several times daily, for example 2, 3, 4 or 8 times, giving for example, 1, 2 or 3 doses each time.

Aerosol combination formulations for treatment of the conditions referred to above in the average adult human are preferably arranged so that each metered dose or “puff” of aerosol contains from about 0.01 mg to about 100 mg of the active compound of this invention, preferably from about 1 mg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of an ERK kinase inhibitor, preferably from about 1 mg to about 200 mg of p38 kinase inhibitor. Administration may be several times daily, for example 2, 3, 4 or 8 times, giving for example, 1, 2 or 3 doses each time.

The following Examples illustrate the preparation of the compounds of the present invention. Melting points are uncorrected. NMR data are reported in parts per million (d) and are referenced to the deuterium lock signal from the sample solvent (deuteriochloroform unless otherwise specified). Mass Spectral data were obtained using a Micromass ZMD APCI Mass Spectrometer equipped with a Gilson gradient high performance liquid chromatograph. The following solvents and gradients were used for the analysis. Solvent A; 98% water/2% acetonirile/0.01% formic acid and solvent B; acetonitrile containing 0.005% formic acid. Typically, a gradient was run over a period of about 4 minutes starting at 95% solvent A and ending with 100% solvent B. The mass spectrum of the major eluting component was then obtained in positive or negative ion mode scanning a molecular weight range from 165 amu to 1100 amu. Specific rotations were measured at room temperature using the sodium D line (589 nm). Commercial reagents were utilized without further purification. THF refers to tetrahydrofuran. DMF refers to N,N-dimethylformamide. Chromatography refers to column chromatography performed using 32-63 mm silica gel and executed under nitrogen pressure (flash chromatography) conditions. Room or ambient temperature refers to 20-25° C. All non-aqueous reactions were run under a nitrogen atmosphere for convenience and to maximize yields. Concentration at reduced pressure means that a rotary evaporator was used.

One of ordinary skill in the art will appreciate that in some cases, protecting groups may be required during preparation. After the target molecule is prepared, the protecting group can be removed by methods well known to those of ordinary skill in the art, such as described in Greene and Wuts, Protective Groups in Organic Synthesis, (2^{nd} Ed., John Wiley & Sons, 1991).

### EXAMPLE 1

### 5-bromo-pyridin-2-yl-hydrazine

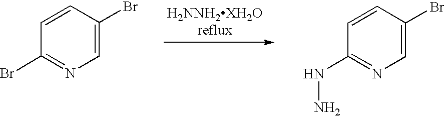

A 12L three-necked round-bottomed flask equipped with a mechanical stirrer and a condenser, connected on top with a nitrogen bubbler and a thermometer, was charged with 2,5-dibromopyridine (442 g, 1.87moles), hydrazine hydrate (55% wt., 1057 ml, 18.7 moles), poly(ethylene glycol) (average Mₙ about 300, 1.87 L), 2-butanol (373 ml) and water (1.87 L). The mixture was heated at reflux for 29 hours. The heating source was removed and the mixture was stirred for an additional 20 hours. To the resulting slurry, cold water (2.2 L) was added. The slurry was stirred for an additional 30 minutes and filtered. The cake was washed with cold water (3×200 ml) and dried in a vacuum-oven (40° C.) for 48 hours. The title compound was obtained as off-white flakes (305 g, yield 87%).

GCMS(m/z): 187 (M+). H¹NMR (400 MHz, CDCl₃): δ 8.14 (d, J=2.0 Hz, 1H), 7.55 (dd, J=8.7/2.0 Hz, 1H), 6.66 (d, J=8.7 Hz, 1H), 5.89 (brs, 1H), 3.65 (brs, 2H).

### EXAMPLE 2

### 6-bromo-3-isopropyl-[1,2,4]triazolo(4,3-a) pyridine Hydrochloride

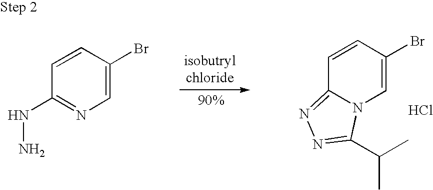

A 500 ml three-necked round-bottomed flask equipped with a mechanical stirrer and a condenser, connected on top to a nitrogen bubbler and a thermometer, was charged with 5bromo-pyridin-2-yl-hydrazine (43.4 g, 0.231 moles) and isobutyryl chloride (218 ml, 2.08 moles). The mixture was gently refluxed for 3 hours. The heating source was then replaced with an ice-water bath and the slurry cooled to room temperature. Hexane (220 ml) was added and the slurry stirred at room temperature for 15 minutes and filtered. The cake was washed with hexane (3×70 ml) and then dried in a vacuum-oven (35° C.) for 48 hours. The title compound was obtained as an off-white powder (58.96 g, yield 92.3%).

### EXAMPLE 3

### 6-bromo-3-isopropyl-[1,2,4]triazolo(4.3-a)pyridine

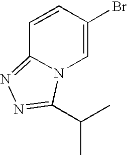

A 5L three-necked round-bottomed flask, equipped with a mechanical stirrer and a thermometer, was charged with 6-bromo-3-isopropyl-[1,2,4]triazolo(4,3-a)pyridine hydrochloride (587.0 g, 2.12 moles), water (1.2 L) and dichloromethane (1.8 L). The biphasic mixture was cooled to 5 to 10° C. using an ice-water bath. Sodium hydroxide (1N aqueous solution) (2.15 L) was added over a period of 10 minutes. The mixture was stirred in the bath for 15 minutes. The organic layer was then isolated and the aqueous layer extracted with dichloromethane (600 mL). The combined organic extracts are washed with 1:1 brine-water (2 L) and dried (MgSO₄). Most of dichloromethane was removed by rotary evaporation. Ethyl acetate (800 ml) was then added. After removing about 400 ml of solvents, hexane (3.2 L) was added. The slurry was stirred in an ice-water bath for 2 hours and then filtered. The cake was washed with 9:1 hexane-ethyl acetate (3×150 ml) and dried in a vacuum-oven (30-35° C.) for 18 hours. The title compound (471.6 g, yield 92.5%), was obtained as a tan sandy powder.

H¹ NMR (400 MHz, CDCl₃): δ 8.06 (s, 1H), 7.64 (d, J=9.5 Hz, 1H), 7.24 (d, J=9.5 Hz, 1H), 3.33 (m, J=7.0 Hz, 1H), 1.52 (d, J=7.0 Hz, 6H).

### EXAMPLE 4

### 3-isopropyl-[1,2,4]triazolo(4.3-a)-6-pyridinecarboxaldehyde

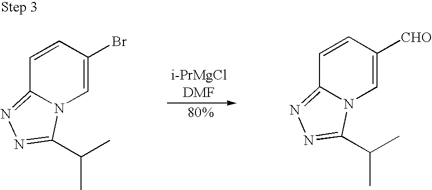

A 12L three-necked round-bottomed flask, equipped with a mechanical stirrer, an addition funnel and a thermometer, was charged with 6-bromo-3-isopropyl-[1,2,4]triazolo(4,3-a)pyridine (200.0 g, 0.833 moles) and tetrahydrofuran (J. T. Baker, low water 2.0 L). The solution was cooled to −8° C. using an acetone/dry ice bath. A solution of isopropylmagnesium chloride in tetrahydrofuran (2.0M, 500 ml, 1.0 mole) L) was added via the addition funnel over a period of 55 minutes. The resulting brownish slurry was stirred between −4 to 0° C. for 30 minutes. Dimethylformamide (Aldrich, anhydrous, 155 ml, 2.0 moles) was added via an addition funnel over a period of 5 minutes. The cooling bath was replaced with a heating mantle and the addition funnel was replaced with a condenser. The slurry was heated to 55° C. and stirred at this temperature for 2 hours. The reaction mixture was cooled to 15° C. and dichloromethane (3 L) was added. The slurry was slowly poured into a stirred and ice-water cooled (15° C.) 10% by weight aqueous solution of citric acid (3 kg) over a period of 5 minutes. The biphasic mixture was stirred at 17 to 20° C. for 30 minutes. The organic layer was then isolated and the aqueous layer extracted with dichloromethane (5×1L). The combined organic extracts were washed with 1:1 v/v brine-water (2 L), dried (MgSO₄) and concentrated. To the brownish residual solid was added ethyl acetate (800 ml). The slurry was stirred at room temperature for 10 minutes at which time hexane (800 ml) was added. The slurry was stirred at room temperature for 2 more hours and filtered. The cake was washed with 1:1 v/v hexane-ethyl acetate (3×150 ml) and dried in a vacuum-oven (30-35° C.) for 18 hours. The title compound was obtained as a yellowish sandy powder (126.6 g, yield 80%).

GCMS(m/z): 189 (M+). H¹ NMR (400 MHz, CDCl₃): δ 10.00 (s, 1H), 8.49 (s, 1H), 7.79 (d, J=9.5 Hz, 1H), 7.68 (d, J=9.5 Hz, 1H), 3.47 (m, J=7.0 Hz, 1H), 1.56 (d, J=7.0 Hz, 6H).

### EXAMPLE 5

### P-toluenesulfinic Acid

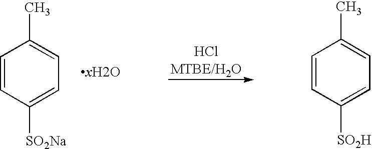

A 5L three-necked round-bottomed flask, equipped with a mechanical stirrer and a thermometer, was charged with p-toluenesulfinic acid, sodium salt hydrate (Aldrich, CH₃C₆H₄SO₂Na.xH₂O, 392.0 g), tap water (2L) and methyl t-butyl ether (2L). The mixture was stirred at room temperature for 10 minutes at which time hydrochloric acid (37% wt. in water, 142 ml, 1.2 moles) was added over a period of 5 minutes. The biphasic mixture was stirred at room temperature for 30 minutes. The organic layer was then isolated and the aqueous layer extracted with methyl t-butyl ether (500 mL). The combined organic extracts were concentrated to a residual white semi-solid, which was diluted with toluene (700 ml). Most of solvents were removed and hexane (1.8L) was then added. The slurry was stirred at room temperature for 30 minutes and filtered. The cake was washed with hexane (2×300 ml) and dried in a vacuum-oven (30-35° C.) for 3 hours. The product, p-toluenesulfinic acid (240.0 g,), was obtained as a white powder.

### EXAMPLE 6

### N-[(2,5-difluoro-phenyl)-(toluene-4-sulfonyl)-methyl]-formamide

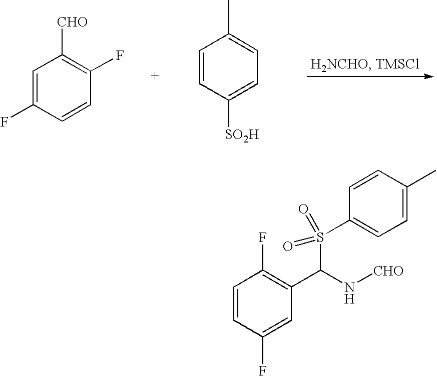

A 5L three-necked round-bottomed flask, equipped with a mechanical stirrer, a condenser and a thermometer, was charged with 2,5-difluorobenzaldehyde (142.11 g, 1 mole). Toluene (500 ml), acetonitrile (500 ml), formamide (99.3 ml, 2.5 moles) and chlorotrimethylsilane (139.6 ml, 1.1 moles) were added respectively. The cloudy mixture was heated to 50° C. and stirred at this temperature for 7 hours. p-Toluenesulfinic acid (218.68 g, 1.4 moles) was added. The mixture was stirred at 50° C. for 6 hours and then 13 hours at room temperature. Methyl t-butyl ether (1.8 L) and water (1.7 L) were then added. The mixture was stirred at room temperature for 15 minutes at which time the organic layer was separated. The aqueous layer was extracted with methyl t-butyl ether (500 ml). Most of the solvents were removed from the combined organic extracts. To the residual white semi-solid, hexane (1L) and water (1 L) were added. The slurry was stirred at room temperature for 30 minutes and filtered. The cake was washed with hexane (2×200 ml) and dried in a vacuum-oven (30° C.) for 18 hours. The product, N-[(2,5-Difluoro-phenyl)-(toluene-4-sulfonyl)-methyl]-formamide (258.3 g, yield 79%,), was obtained as a white powder.

### EXAMPLE 7

### [α-(p-toluenesulfonyl)-2,5-difluorobenzyl]isonitrile

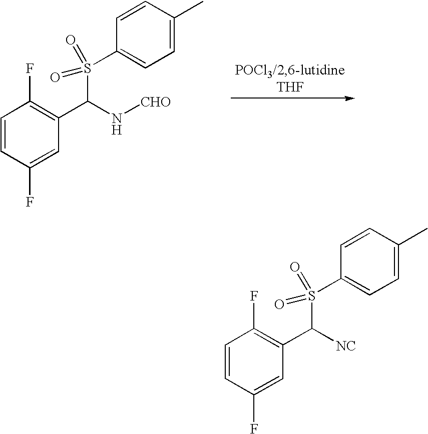

A 5L three-necked round-bottomed flask, equipped with a mechanical stirrer, an addition funnel and a thermometer, was charged with N-[(2,5-Difluoro-phenyl)-(toluene-4-sulfonyl)-methyl]-formamide (207.0 g, 0.636 moles) and tetrahydrofuran (J. T. Baker, low water, 1.5 L). Phosphorous oxychloride (118.6 ml, 1.27 moles) was quickly poured into the reaction mixture (less than 5 minutes). The mixture was stirred at room temperature for 10 minutes and then cooled to 4° C. using an ice/water bath. 2,6-Lutidine (445 ml, 3.82 moles) was added via the addition funnel over a period of 30 minutes. The cooling bath was then removed and the mixture was stirred at room temperature for 18 hours. The reaction mixture was poured into a stirred and ice-water cooled solution of 1.5 kg of ice and 1.1 L of saturated aqueous sodium bicarbonate (NaHCO₃). The mixture was then extracted with ethyl acetate (2L plus 1.5 L). The combined organic extracts were washed with 1N aqueous hydrochloric acid (3 L), saturated aqueous NaHCO₃ (3L) and brine (3L); and then dried (MgSO4). After removing all solvents, isopropanol (1.8 L) was added to the residual brownish solid. The resulting slurry was stirred at room temperature for 2 hours. Water (0.9 L) was added and the slurry was stirred for additional 30 minutes at room temperature and then filtered. The cake was washed with 2:1 isopropanol-water (2×500 ml) and dried in a vacuum-oven (30° C.) for 48 hours. The product, [α-(p-Toluenesulfonyl)-2,5-difluorobenzyl]isonitrile (133.4 g, yield 68%,), was obtained as a brownish powder.

H¹NMR (400 MHz, CDCl₃): δ, 7.7 (d, J=8.3 Hz, 2H) 7.41 (d, J=8.3 Hz, 2H), 7.18 (m, 3H), 5.91 (s, 1H), 2.50 (s. 3H).

### EXAMPLE 8

### [α-(p-toluenesulfonyl)-2,5-difluorobenzyl]isonitrile

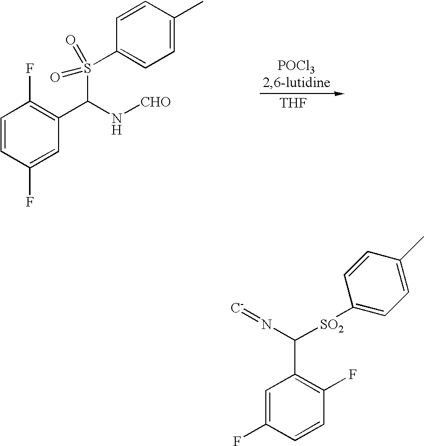

To a clean a dry nitrogen purged acetone boiled out 100 gallon glass lined reactor was charged, 7.9 Kg of N-[(2,5-Difluoro-phenyl)-(toluene-4-sulfonyl)-methyl]-formamide (24, moles), 16 gallons of tetrahydrofuran and 7.8 Kg of phosphorous oxychloride (51 moles). The batch was allowed to stir at 20° C. for 30 minutes and then cooled to 3.5° C. To the batch was added 15.8 Kg of 2,6-lutidine (146 moles) over 15 minutes. The reaction mixture was allowed to warm to 23° C. and was stirred for 17 hours at 23° C. The reaction was judged complete by HPLC and was charged to a 40 gallon solution of 10% sodium bicarbonate at 22° C., and the contents were allowed to stir for 30 minutes. To the batch was then added 25 gallons of ethyl acetate and the layers were separated. The water layer was backwashed with 9 gallons of ethyl acetate and the product rich ethyl acetate combined with the first wash. The product rich ethyl acetate layers were added to a 10% citric acid solution (20 gallons) and then stirred. The organic layer was checked by HPLC for 2,6 lutidine and then separated. The organic layer was washed with 10 gallons of saturated NaCl and dried over 7.9 Kg of magnesium sulfate. The drying agents were removed by filtration and the cake was washed with 4 gallons of ethyl acetate. The ethyl acetate layer was concentrated to 7 gallons under vacuum at an internal temperature of 24° C. The batch was then added to 11 gallons of IPO at 21° C. and allowed to granulate at 4° C. for 12 hours. The product was isolated via filtration and washed with 4 gallons of 5° C. IPO. The product was then dried at 34° C. for 22 hours with nitrogen bleed to recover 5.0 Kg of the title compound (66% yield).

### EXAMPLE 9

### 6-[4-(2,5-difluoro-phenyl)-oxazol-5-yl]-3-isopropyl-[1 2,4]triazolo-[4,3-a]pyridine

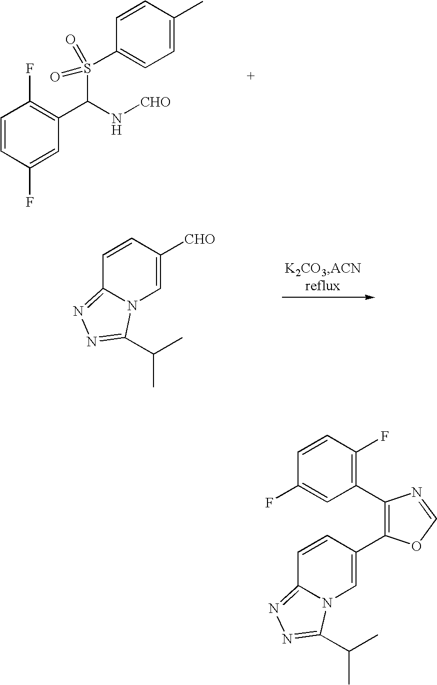

A 5L three-necked round-bottomed flask, equipped with a mechanical stirrer, a condenser and a thermometer, was charged with [α-(p-Toluenesulfonyl)-2,5difluorobenzyl]isonitrile (179.4 g, 0.584 moles), 3-isopropyl-[1,2,4]triazolo(4,3-a)-6pyridinecarboxaldehyde (110.46 g, 0.584 moles), potassium carbonate (Aldrich, <325 mesh, 104.88 g, 0.759 moles) and acetonitrile (1.75 L). The mixture was heated at reflux and stirred for 22 hours. The reaction mixture was then cooled to room temperature and poured into a stirred solution of 2 kg of ice and 5 kg of water. The resulting slurry was stirred at room temperature for 2 hours and filtered. The brownish solid was washed with water (2×500 ml) and dried in a vacuum-oven (30° C.) for 48 hours. The crude product (180 g) was purified over a silica gel column (1.1 kg) and eluted with 1:1 ethyl acetate-hexane (to remove less polar impurities), ethyl acetate and finally 20:1 ethyl acetate-methanol. The fractions containing mainly the product were combined and concentrated to small volume (about 600 ml). The resulting slurry was filtered. The cake was washed with ethyl acetate and dried in a vacuum-oven (30° C.) for 18 hours. The light brownish powder (142 g) was further purified by recrystallization from isopropanol (800 ml). 6-[4-(2,6-Difluoro-phenyl)-oxazol-5-yl]-3-isopropyl[1,2,4]triazolo[4,3-a]pyridine was obtained as a light-tan powder (142.1 g, yield 61%).

Melting point 175.7-176.2° C. Elemental analysis, found: C 63.54%, H 4.08%, N 16.56 ; Analytical calculated for: C 63.52%, H 4.15%, N 16.46%. LCMS (m/z): 341 (M+1). ¹HNMR (400 MHz, CDCl₃): δ 8.18 (s, 1H), 8.12 (s, 1H), 7.89 (d, 1H, J=9.6 Hz), 7.46-7.51 (m, 1H), 7.37 (d, 1H J=9.6 Hz), 7.05-7.1 (m, 2H), 3.30-3.33 (m, 1H), 1.48 (d, 6H, J=7.1 Hz).

### EXAMPLE 10

### 6-[4-(2,5-difluoro-phenyl-oxazol-5-yl]-3-isopropyl-[1,2,4]triazolo[4,3-a]pyridine Hydrogen Chloride

Crude 6-[4-(2,5-difluoro-phenyl)-oxazol-5-yl]-3-isopropyl-[1,2,4]triazolo[4,3-a]pyridine (5.0 g) was dissolved in isopropanol (40 ml). Hydrochloric acid (13.3% weight) in isopropanol (4.4 g) was added. The resulting slurry was stirred at room temperature for 30 minutes and filtered. The cake was washed with isopropanol and dried in a vacuum oven (80° C.) for 2 hours. 6-[4-(2,5-Difluoro-phenyl)-oxazol-5-yl]-3-isopropyl-[1,2,4]triazolo[4,3-a]pyridine hydrogen chloride was obtained as an off-white solid (2.8 g, yield 50%).

¹HNMR (400 MHz, CDCl₃): δ 8.49 (d, J=9.5 Hz, 1H), 8.38 (s, 1H), 8.16 (s, 1H), 7.90 (d, J=9.5 Hz, 1H), 7.49-7.53 (m, 1H), 7.13-7.23 (m, 2H), 3.43-3.50 (m, 1H), 1.55 (d, J =7.1 Hz, 6H).

### EXAMPLE 11

### 6-[4-(2,5-difluoro-phenyl)-oxazol-5-yl]-3-isopropyl-[1,2,4]triazolo-[4,3a]pyridine Methanesulfonate

6-[4-(2,5-Difluoro-phenyl]-oxazo[-5-yl]-3-isopropyl-[1,2,4]triazolo[4,3-a]pyridine (5.10 g, 15 mmol) was dissolved in isopropanol (25 ml). A solution of methanesulfonic acid (1.44 g, 15 mmol) in isopropanol (15 ml) was added. The resulting slurry was stirred at room temperature for 3 hours and filtered. The cake was washed with isopropanol and dried in a vacuum oven (80° C.) for 4 hours. 6-[4-(2,5-Difluoro-phenyl)-oxazol-5-yl]-3-isopropyl-[1,2,4]triazolo[4,3-a]pyridine methanesulfonate was obtained as an off-white powder (6.03 g, yield 92%).

¹HNMR (400 MHz, CDCl₃): δ 8.67 (d, J=9.5 Hz, 1H), 8.38 (s, 1H), 8.15 (s, 1H), 7.83 (d, J=9.5 Hz, 1H), 7.46-7.50 (m, 1H), 7.13-7.22 (m, 2H), 3.44-3.51 (m, 1H), 2.86 (s, 3H), 1.54 (d, J =7.1 Hz, 6H).

### EXAMPLE 12

### 6-[4-(2,5-difluoro-phenyl)-oxazol-5-yl]-3-isopropyl-[1,2,4]triazolo-[4,3-a]pyridine p-toluenesulfonate

To 6-[4-(2,5-Difluoro-phenyl)-oxazol-5-yl]-3-isopropyl-[1,2,4]triazolo[4,3-a]pyridine (5.0 g, 15 mmol) slurried in acetone (50 ml) was added p-Toluenesulfonic acid (2.7g, 15 mmol). The resulting slurry was heated to 50° C. to form a solution and was then cooled and stirred at room temperature for 12 hours and filtered. 6-[4-(2,5-Difluoro-phenyl)-oxazol-5-yl]-3-isopropyl-[1,2,4]triazolo[4,3-a]pyridine p-toluenesulfonate was obtained.

### EXAMPLE 13

### 6-[4-(2,5-difluoro-phenyl)-oxazol-5-yl]-3-isopropyl-[1,2,4]triazolo-[4,3-a]pyridine Sulfate

To 6-[4-(2,-Difluoro-phenyl)-oxazo-5-yl]-3-isopropyl-[1,2,4]triazolo[4,3-a]pyridine (5.0 g, 15 mmol) slurried in acetone (50 ml) was added sulfuric acid (850 μl). The resulting slurry was heated to reflux to form a solution and was then cooled and stirred at room temperature for 12 hours and filtered to yield 4.2 grams of 6-[4-(2,5-difluoro-phenyl)-oxazol-5-yl]-3-isopropyl-[1,2,4]triazolo[4,3-a]pyridine p-toluenesulfate.

### EXAMPLE 14

### 6-[oxazol-5-yl]-3-isopropyl-[1,2,4]triazolo[4,3-a]pyridine

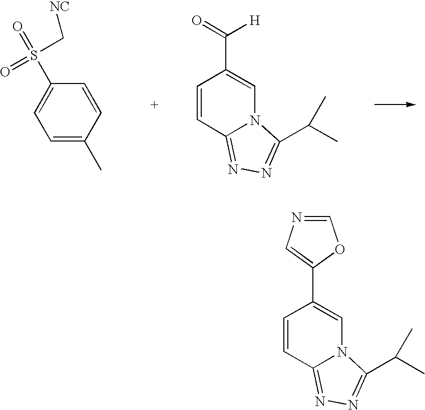

To a clean dry 5 liter round bottomed flask equipped with a mechanical stirrer, nitrogen bubbler, heating mantle, temperature controller, and condenser, was charged 3-isopropyl-[1,2,4]triazolo(4,3-a)-6-pyridinecarboxaldehyde (140.9 grams, 0.745 moles), potassium carbonate (133.8 grams, 0.968 moles), tosylmethyl isocyanide (146.9 grams, 0.745 moles),and methanol (2114 ml). This mixture was heated at reflux and stirred for 1.5 to 2.0 hours at 65 to 70° C. Assay by HPLC showed the reaction to be complete. The pot was concentrated atmospherically to about one third of original volume. Water (1409 ml), was added and the pot further concentrated to a pot temperature of 65 to 66° C. to remove the remaining methanol. After cooling, the desired product was extracted with methylene chloride (1409 ml). The extraction was repeated twice with methylene chloride (2 times 705 ml). The combined extracts were atmospherically concentrated and displaced with Isopropyl alcohol (420 ml). A thick slurry formed. Hexanes (1690 ml) were added and the slurry allowed to granulate for 12 to 16 hours at 20 to 25° C. The solids were collected by vacuum filtration, washed with hexanes, and dried to yield 111.45 grams, 97.8% purity (HPLC), 65.5% of theory.

¹H NMR (CDCl₃, 400 MHz) δ 8.23 (s, 1H), 7.98 (s, 1H), 7.82 (d, 1H, J=9.5 Hz), 7.46-7.43 (m, 2H), 3.43 (sept, 1H, J=7.05 Hz), 1.56 (d, 6H, J=9.05 Hz); MS 229 (M⁺+1)

### EXAMPLE 15

### 6-[4-bromo-oxazol-5-yl]-3-isopropyl-[1,2,4]triazolo[4,3-a]pyridine

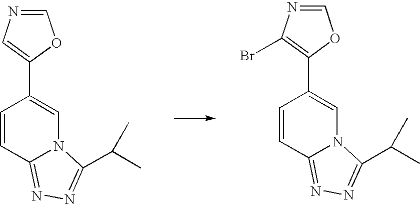

A clean, dry, 1 liter 4 neck round bottom flask equipped with mechanical stirrer, temperature probe, and purged with nitrogen, was charged with 6-[oxazol-5-yl]-3-isopropyl-[1,2,4]triazolo[4,3-a]pyridine (45.2 grams 0.198 moles) and N,N-dimethylformamide (271 ml). The pot was cooled below −60° C. with a dry ice/acetone bath. Lithium bis(trimethylsilyl)amide, 1 molar solution in tetrahydrofuran (198 ml 0.198 moles), was added, keeping the temperature below −60° C. After the addition was complete, the pot was further cooled to below −70° C. and stirred for 1 hour. While stirring, a solution of N-bromosuccinimide (35.24 g 0.198 moles) and N,N-dimethylformamide (105 ml), were stirred in a separate 500 ml round bottom flask under nitrogen. After the one hour stir at −70° C., the solution of N-bromosuccinimide and N,N-dimethylformamide was slowly added to the anion keeping the temperature below −70° C. After the addition, the reaction was continued for one hour below −70° C. The batch was then warmed to room temperature and quenched into methylene chloride (452 ml) and 1N sodium hydroxide (452 ml). The organic layer was then separated. The aqueous layer was extracted a second time with methylene chloride (135 ml). The combined organic phase was washed with 1N sodium hydroxide (452 ml) and saturated brine solution (452 ml). The organic phase was then dried over magnesium sulfate (50 grams) and concentrated/displaced with isopropyl ether (226 ml) to a temperature of 42° C. A thick slurry formed upon cooling. The solids were granulated at 20 to 25° C. for two hours, filtered, washed with isopropyl ether (50 ml), and dried to afford 53.0 grams of light yellow solids, 96.4% purity (HPLC), 87% of theory.

¹H NMR (CDCl₃, 400 MHz) δ 8.56 (s, 1H), 7.95 (s, 1H), 7.85 (d, 1H, J=9.5 Hz), 7.77 (d, 1H, J=9.5 Hz), 3.43 (sept, 1H, J=7.05 Hz), 1.56 (d, 6H, J=7.05 Hz); MS: 310, 309, 308, 307 (M⁺+1).

### EXAMPLE 16

### 3-isopropyl-6-[4-(2,5-difluoro-phenyl)-oxazol-5-yl]-[1,2,4]triazolo-[4,3-a]pyridine

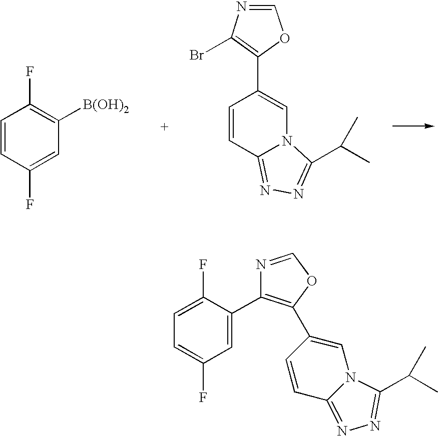

6-[4-bromo-oxazol-5-yl]-3-isopropyl-[1,2,4]triazolo[4,3-a]pyridine (33.0 grams, 0.107 moles), difluorophenylboronic acid (25.34 grams, 0.1605 moles), Pd(PPh₃)₄ (12.36 grams, 0.0107 moles), triethylamine (22.37 ml, 0.1605 moles), 2B ethanol (495 ml) and water (33 ml), were added to a 2 liter 4 neck round bottom flask (equipped with mechanical stirring, nitrogen, heating mantle, temperature controller, and a condenser). The batch was stirred while heating to 65 to 70° C. The reaction was stirred overnight at about 70° C. Additional difluorophenylboronic acid (8.5 grams, 0.054 moles) and triethylamine (7.53 ml, 0.054 moles), were added and the reaction was allowed to proceed overnight at 70° C. Additional difluorophenylboronic acid (8.5 grams, 0.054 moles) and triethylamine (7.53 ml, 0.054 moles), were added and the reaction was allowed to proceed overnight once again at 70° C. Toluene (30 ml) was added and the reaction was allowed to go overnight once again at 70° C. The reaction sample showed no more starting material by HPLC. Water (495 ml) was added to the batch and the pot granulated for 4 hours at 20 to 25° C. The solids were collected by vacuum filtration, washed with 2B ethanol/water 50:50 (25 ml of each), and dried in a vacuum oven at 45° C. for 4 hours under full vacuum to afford 14.4 grams of the title compound (40.6% yield, 93.4% purity by HPLC).

### EXAMPLE 17

### 3-isopropyl-6-[4-(2,5-difluoro-phenyl)-oxazol-5-yl]-[1,2,4]triazolo-[4,3-a]pyridine

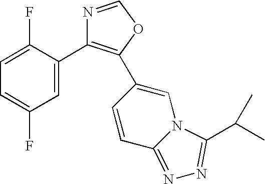

Crude 3-isopropyl-6-[4-(2,5-difluoro-phenyl)-oxazol-5-yl]-[1,2,4]triazolo[4,3-a]pyridine (5.0 grams), Darco G-60 carbon (500 mg), and isopropyl alcohol (30 ml), were heated to 80° C. in a single neck 100 ml round bottom flask. The solution was allowed to cool to 60° C. and filtered over Filter-aid® to remove carbon. The cake was washed with isopropyl alcohol (30 ml), then allowed to further cool to 20 to 25° C. and granulate overnight. The solids were collected by vacuum filtration, washed with isopropyl alcohol (10 ml), and dried to afford 4.2 grams of the title compound, 98.8% purity (HPLC), 84% yield.

### EXAMPLE 18

### 3-isopropyl-6-[4-(2,5-difluoro-phenyl)-oxazol-5-yl]-[1,2,4]triazolo-[4,3-a]pyridin

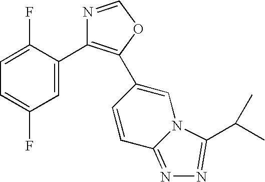

Pure 3-isopropyl-6-[4-(2,5-difluoro-phenyl)-oxazol-5-yl]-[1,2,4]triazolo[4,3-a]pyridine (3.4 grams), and acetone (41 ml) were heated to 50 to 55° C. until a clear golden solution was achieved. The heat was removed and the solution was allowed to cool, (approximately 35 to 40° C.), and granulate overnight at 20 to 25° C. The solids were collected by vacuum filtration, washed with acetone (7 ml), and dried to afford 2.38 grams of crystal form B, 99.6% purity (HPLC), 70% yield.

1. A process for preparing a compound of the formula 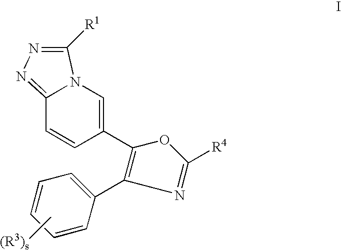
wherein R¹ is selected from the group consisting of hydrogen, —C≡N, (C₁-C₆)alkyl, (C₂-C₆)alkenyl, (C₂-C₆)alkynyl, (C₃-C₁₀)cycloalkyl, phenyl, (C₁-C₁₀)heteroaryl, (C₁-C₁₀)heterocyclic and (R²)₂—N—;wherein each of the aforesaid (C₁-C₆)alkyl, (C₃-C₁₀)cycloalkyl, phenyl, (C₁-C₁₀)heteroaryl and (C₁-C₁₀)heterocyclic substituents may optionally be independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, (C₁-C₆)alkyl-O—(C═O)—, [(C₁-C₆)alkyl]₂-N—(C═O)—, phenyl-[((C₁-C₆)alkyl)-N]—(C═O)—, —NO₂, [(C₁-C₆)alkyl]₂-amino, (C₁-C₆)alkyl-(C═O)—[((C₁-C₆)alkyl)-N]—, phenyl-(C═O)—[((C₁-C₆)alkyl)-N]—, [(C₁-C₆)alkyl-]₂N—(C═O)—[((C₁-C₆)alkyl)-N]—, (phenyl-)₂N—(C═O)—[((C₁-C₆)alkyl)-N]—, (C₁-C₆)alkyl-O—(C═O)—[((C₁-C₆)alkyl)-N]—, phenyl-O—(C═O)—[((C₁-C₆)alkyl)-N]—, (C₁-C₆)alkyl-SO₂—, phenyl-SO₂—, (C₁-C₆)alkoxy, perhalo(C₁-C₆)alkoxy, phenoxy, (C₁-C₆)alkyl-(C═O)—O—, phenyl-(C═O)—O—, [(C₁-C₆)alkyl-]₂N—(C═O)—O—, (phenyl-)₂N—(C═O)—O—; wherein when said R² phenyl contains two adjacent substituents, such substituents may optionally be taken together with the carbon atoms to which they are attached to form a five to six membered carbocyclic or heterocyclic ring; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy;
each R² is independently selected from hydrogen, (C₁-C₆)alkyl, phenyl, (C₁-C₁₀)heteroaryl, (C₁-C₁₀)heterocyclic and (C₃-C₁₀)cycloalkyl; wherein each of the aforesaid R² (C₁-C₆)alkyl, phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₂)heteroaryl, (C₁-C₁)heterocyclic, (C₃-C₁₀)cycloalkyl, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, —NO₂, [(C₁-C₆)alkyl]₂-amino, (C₁-C₆)alkyl-(C═O)—[((C₁-C₆)alkyl)-N]—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, (C₁-C₆)alkyl-O—(C═O)—, [(C₁-C₆)alkyl]₂-N—(C═O)—, phenyl-[((C₁-C₆)alkyl)-N]—(C═O)—, (C₁-C₆)alkyl-(C═O)—O— and phenyl-(C═O)—O—; wherein two R² (C¹-C₆)alkyl groups may be taken together with the nitrogen atom to which they are attached to form a five to six membered heterocyclic or heteroaryl ring;
each R³ is independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)— and (C₁-C₆)alkyl-(C═O)—O—; wherein two adjacent R³ substituents may be optionally taken together with the carbon atoms to which they are attached to form a five to six membered carbocyclic or heterocyclic ring;
s is an integer from zero to five;
R⁴ is selected from the group consisting of hydrogen, fluoro, chloro or R⁵—B—(CH₂)ₙ—;
n is an integer from zero to six;
each B is independently a bond, —(CHR⁶)—, —O—, —S—, —(SO₂)—, —(C═O)—, —O—(C═O)—, —(C═O)—O—, —(C═O)—NR⁶—, —(R⁶—N)—, —(R⁶—N)—SO₂—, —(R⁶—N)—(C═O)—, —SO₂—(NR⁶)—, —(R⁶—N)—(C═O)—(NR⁷)—, —(O)—(C═O)—(NR⁶)— or —(R⁶—N—(C═O)—O—;
R⁵ is selected from the group consisting of hydrogen, —CF₃, —C≡N, R⁹—(R⁸CH)ₘ—, phenyl, (C₁-C₁₀)heterocyclic, (C₁-C₁₀)heteroaryl, and (C₃-C₁₀)cycloalkyl; wherein each of the aforesaid R⁵ phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁₃ -C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—; wherein two adjacent R⁵ substituents of said phenyl, (C₁-C₁₀)heteroaryl, (C₁-C₁₀)heterocyclic and (C₃-C₁₀)cycloalkyl may optionally be taken together with the carbon or heteroatom to which they are attached to form a five or six membered carbocyclic or heterocyclic ring;
m is an integer from one to six;
R⁶ is hydrogen, (C₁-C₆)alkyl-SO₂— or (C₁-C₆)alkyl;
R⁷ is hydrogen or (C₁-C₆)alkyl;
each R⁸ is independently selected from the group consisting of hydrogen, amino, (C₁-C₆)alkoxy and (C₁-C₆)alkyl;
R⁹ is selected from the group consisting of hydrogen, (C₁-C₆)alkyl, (C₂-C₆)alkenyl, (C₂-C₆)alkynyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, phenyl-SO₂—NH—, (C₁-C₆)alkyl-SO₂—[((C₁-C₆)alkyl)-N]—, phenyl-SO₂—[((C₁-C₆)alkyl)-N]—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—;
or an acceptable salt thereof; comprising reacting a compound of the formula 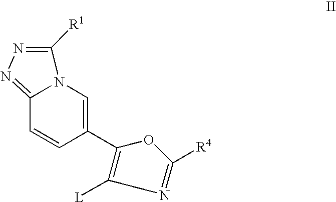
wherein L is a leaving group and R¹ and R⁴ are as defined above, with a compound of the formula 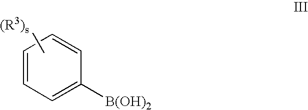
wherein R³ and s are as defined above and a transition metal catalyst.

2. A process according to claim 1, where the reaction is performed in the presence of toluene.

3. A process for preparing a compound of the formula 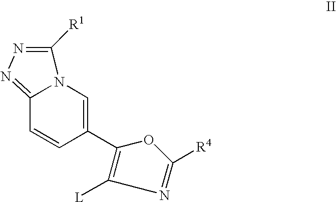
wherein L is halo and R¹ and R⁴ are as defined above;
R¹ is selected from the group consisting of hydrogen, —C≡N, (C₁-C₆)alkyl, (C₂-C₆)alkenyl, (C₂-C₆)alkynyl, (C₃-C₁₀)cycloalkyl, phenyl, (C₁-C₁₀)heteroaryl, (C₁-C₁₀)heterocyclic and (R¹)₂—N—; wherein each of the aforesaid (C₁-C₆)alkyl, (C₃-C₁₀)cycloalkyl, phenyl, (C₁-C₁₀)heteroaryl and (C₁-C₁₀)heterocyclic substituents may optionally be independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, —NO₂, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, phenyl-HN—(C═O)—NH—, (phenyl-)₂N—(C═O)—NH—, phenyl-HN—(C═O)—[((C₁-C₆)alkyl)-N]—, (phenyl-)₂N—(C═O)—[((C₁-C₆)alkyl)-N]—, (C₁-C₆)alkyl-O—(C═O)—NH—, (C₁-C₆)alkyl-O—(C═O)—[((C₁-C₆)alkyl)-N]—, phenyl-O—(C═O)—NH—, phenyl-O—(C═O)—[((C₁-C₆)alkyl)-N]—, (C₁-C₆)alkyl-SO₂NH—, phenyl-SO₂NH—, (C₁-C₆)alkyl-SO₂—, phenyl-SO₂—, hydroxy, (C₁-C₆)alkoxy, perhalo(C₁-C₆)alkoxy, phenoxy, (C₁-C₆)alkyl-(C═O)—O—, phenyl-(C═O)—O—, H₂N—(C═O)—O—, (C₁-C₆)alkyl-HN—(C═O)—O—, [(C₁-C₆)alkyl-]₂N—(C═O)—O—, phenyl-HN—(C═O)—O—, (phenyl-)₂N—(C═O)—O—; wherein when said R¹ phenyl contains two adjacent substituents, such substituents may optionally be taken together with the carbon atoms to which they are attached to form a five to six membered carbocyclic or heterocyclic ring; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy;
each R² is independently selected from hydrogen, (C₁-C₆)alkyl, phenyl, (C₁-C₁₀)heteroaryl, (C₁-C₁₀)heterocyclic and (C₃-C₁₀)cycloalkyl; wherein each of the aforesaid R¹ (C₁-C₆)alkyl, phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—; wherein two R² (C₁ -C₆)alkyl groups may be taken together with the nitrogen atom to which they are attached to form a five to six membered heterocyclic or heteroaryl ring;
R⁴ is selected from the group consisting of hydrogen, fluoro, chloro or R⁵—B—(CH₂)ₙ—;
n is an integer from zero to six;
each B is independently a bond, —(CHR⁶)—, —O—, —S—, —(SO₂)—, —(C═O)—, —O—(C═O)—, —(C═O)—O—, —(C═O)—NR⁶—, —(R⁶—N)—, —(R⁶—N)—SO₂—, —(R⁶—N)—(C═O)—, —SO₂—(NR⁶)—, —(R⁶—N)—(C═O)—(NR⁷)—, —(O)—(C═O)—(NR⁶)— or —(R⁶—N)—(C═O)—O—;
R⁵ is selected from the group consisting of hydrogen, —CF₃, —C═N, R⁹—(R⁸CH)ₘ—, phenyl, (C₁-C₁₀)heterocyclic, (C₁-C₁₀)heteroaryl, and (C₃-C₁₀)cycloalkyl; wherein each of the aforesaid R⁵ phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O), (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—; wherein two adjacent R⁵ substituents of said phenyl, (C₁-C₁₀)heteroaryl, (C₁-C₁₀)heterocyclic and (C₃-C₁₀)cycloalkyl may optionally be taken together with the carbon or heteroatom to which they are attached to form a five or six membered carbocyclic or heterocyclic ring;
m is an integer from one to six;
R⁶ is hydrogen, (C₁-C₆)alkyl-SO₂— or (C₁-C₆)alkyl;
R⁷ is hydrogen or (C₁-C₆)alkyl;
each R⁸ is independently selected from the group consisting of hydrogen, amino, (C₁-C₆)alkoxy and (C₁-C₆)alkyl;
R⁹ is selected from the group consisting of hydrogen, (C₁-C₆)alkyl, (C₂-C₆)alkenyl, (C₂-C₆)alkynyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, phenyl-SO₂—NH—, (C₁-C₆)alkyl-SO₂—[((C₁-C₆)alkyl)-N]—, phenyl-SO₂—[((C₁-C₆)alkyl)-N]—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—;
by reaction of a compound of the formula 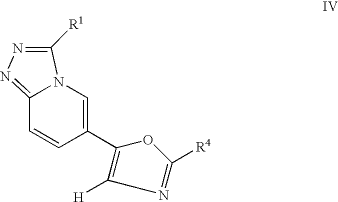
wherein R¹ and R⁴ are as defined above; with a halogenating reagent.

4. A process according to claim 2, wherein reaction is performed in the presence of a strong base.

5. A process according to claim 3, wherein said strong base is lithium bis(trimethylsilyl)amide or lithium diisopropylamide.

6. A process according to claim 4, additionally comprising a polar aprotic solvent.

7. A process according to claim 5, wherein said polar aprotic solvent is N,N-dimethylformamide.

8. A process for preparing a compound of the formula 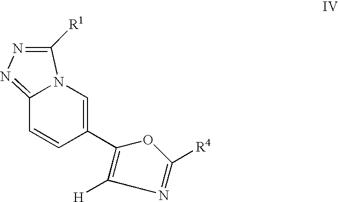
wherein R⁴ is hydrogen and R¹ is as defined above in claim 1; comprising reacting a compound of the formula 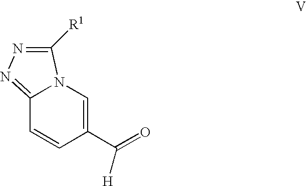
wherein R¹ is as defined above; with tosylmethyl isocyanide and a base.

9. A process for preparing a compound of the formula 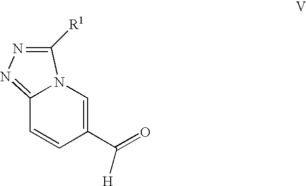
wherein R¹ is as defined above in claim 2; by reaction of a compound of the formula 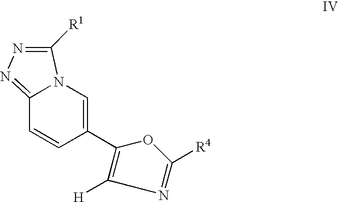
wherein L′ is bromo or iodo and R¹ is as defined above; with an (C₁-C₆)alkyl magnesium halide or (C₁-C₆)alkyl lithium, followed by reaction with a disubstituted formamide reagent;
with the proviso that R¹ is other than isopropyl.

10. A process according to claim 9, additionally comprising citric acid or potassium dihydrogen phosphate.

11. A process for preparing a compound of the formula 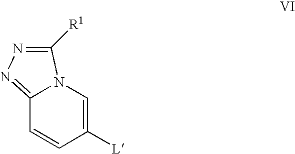
wherein L′ is halo; and R¹ is isopropyl, comprising reacting a compound of the formula 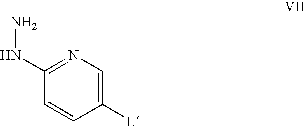
wherein L′ is halo; with isobutyryl chloride.

12. A process for preparing a compound of the formula 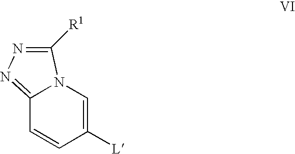
wherein L′ is halo;
R¹ is selected from the group consisting of hydrogen, —C≡N, (C₁-C₆)alkyl, (C₂-C₆)alkenyl, (C₂-C₆)alkynyl, (C₃-C₁₀)cycloalkyl, phenyl, (C₁-C₁₀)heteroaryl, (C₁-C₁₀)heterocyclic and (R¹)₂—N—; wherein each of the aforesaid (C₁-C₆)alkyl, (C₃-C₁₀)cycloalkyl, phenyl, (C₁-C₁₀)heteroaryl and (C₁-C₁₀)heterocyclic substituents may optionally be independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, (C₁-C₆)alkyl-O—(C═O)—, [(C₁-C₆)alkyl]₂-N—(C═O)—, phenyl-[((C₁-C₆)alkyl)-N]—(C═O)—, —NO₂, [(C₁-C₆)alkyl]₂-amino, (C₁-C₆)alkyl-(C═O)—[((C₁-C₆)alkyl)-N]—, phenyl-(C═O)—[((C₁-C₆)alkyl)-N]—, [(C₁-C₆)alkyl-]₂N—(C═O)—[((C₁-C₆)alkyl)-N]—, (phenyl-)₂N—(C═O)—[((C₁-C₆)alkyl)-N]—, (C₁-C₆)alkyl-O—(C═O)—[((C₁-C₆)alkyl)-N]—, phenyl-O—(C═O)—[((C₁-C₆)alkyl)-N]—, (C₁-C₆)alkyl-SO₂—, phenyl-SO₂—, (C₁-C₆)alkoxy, perhalo(C₁-C₆)alkoxy, phenoxy, (C₁-C₆)alkyl-(C═O)—O—, phenyl-(C═O)—O—, [(C₁-C₆)alkyl-]₂N—(C═O)—O—, (phenyl-)₂N—(C═O)—O—; wherein when said R¹ phenyl contains two adjacent substituents, such substituents may optionally be taken together with the carbon atoms to which they are attached to form a five to six membered carbocyclic or heterocyclic ring; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy;
and each R² is independently selected from hydrogen, (C₁-C₆)alkyl, phenyl, (C₁-C₁)heteroaryl, (C₁-C₁₀)heterocyclic and (C₃-C₁₀)cycloalkyl; wherein each of the aforesaid R¹ (C₁-C₆)alkyl, phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, —NO₂, [(C₁-C₆)alkyl]₂-amino, (C₁-C₆)alkyl-(C═O)—[((C₁-C₆)alkyl)-N]—, phenyl-(C═O)—[((C₁-C₆)alkyl)-N]—, —CN, (C₁-C₆)alkyl-(C═O)₁, phenyl-(C═O)—, (C₁-C₁₀)heteroaryl-(C═O)—, (C₁-C₁₀)heterocyclic-(C═O)—, (C₃-C₁₀)cycloalkyl-(C═O)—, (C₁-C₆)alkyl-O—(C═O)—, [(C₁-C₆)alkyl]₂-N—(C═O)—, phenyl-[((C₁-C₆)alkyl)-N]—(C═O)—, (C₁-C₆)alkyl-(C═O)—O— and phenyl-(C═O)—O—; wherein two R² (C₁-C₆)alkyl groups may be taken together with the nitrogen atom to which they are attached to form a five to six membered heterocyclic or heteroaryl ring;
with the proviso that R¹ is other than isopropyl;
comprising reacting a compound of the formula 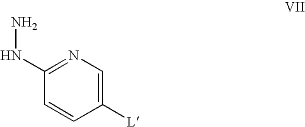
wherein L′ is halo; with a reagent of the formula 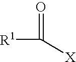
wherein X is halo, tosyl, mesyl or a group of the formula 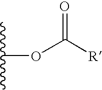
wherein R′ is R¹, t-butyl, or (C₁-C₆)alkyl-O—;
and R¹ is other than isopropyl.

13. A process for preparing a compound of the formula 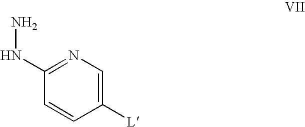
wherein L′ is halo;
comprising reacting a compound of the formula 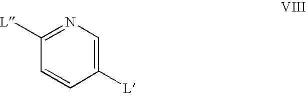
wherein L′ is halo and L″ is halo; with a hydrazine, PEG-300, water and 2-butanonol.

14. A process according to claim 1, wherein R¹ is optionally substituted (C₁-C₆)alkyl, phenyl, (C₃-C₁₀)cycloalkyl, (C₁-C₁₀)heteroaryl or (C₁-C₁₀)heterocyclic.

15. A process according to claim 1, wherein R¹ is (C₁-C₆)alkyl, optionally substituted with one to four groups independently selected from halo, hydroxy, (C₁-C₆)alkyl, (C₂-C₆)alkenyl, (C₂-C₆)alkynyl, (C₁-C₆)alkoxy, perhalo(C₁-C₆)alkyl, perhalo(C₁-C₆)alkoxy, —CN, —NO₂, amino, (C₁-C₆)alkylamino, [(C₁-C₆)alkyl]₂-amino, HO—(C═O)—, (C₁-C₆)alkyl-(C═O)—, (C₁-C₆)alkyl-O—(C═O)—, (C₁-C₆)alkyl-CO₂—, (C₁-C₆)alkyl-(C═O)—NH—, (C₁-C₆)alkyl-NH—(C═O)—, (C₁-C₆)alkyl-(C═O)—[((C₁-C₆)alkyl)-N]—, (C₁-C₆)alkyl-[((C₁-C₆)alkyl)-N]—(C═O)—, (C₁-C₆)alkyl-SO₂NH—, (C₁-C₆)alkyl-SO₂—, optionally substituted phenyl-(C═O)—, optionally substituted phenyl-(C═O)—O—, optionally substituted phenoxy, optionally substituted phenylNH-(C═O)—, optionally substituted phenyl-[((C₁-C₆)alkyl)-N]—(C═O)—, optionally substituted phenyl-(C═O)—NH— and optionally substituted phenyl-(C═O)—[((C₁-C₆)alkyl)-N]—.

16. A process according to claim 1, wherein R¹ is (C₁-C₄)alkyl.

17. A process according to claim 1, wherein R¹ is isopropyl.

18. A process according to claim 1, wherein R¹ is optionally substituted (C₃-C₆)cycloalkyl.

19. A process according to claim 1, wherein R¹ is optionally substituted phenyl.

20. A process according to claim 1, wherein R¹ is optionally substituted phenyl, wherein said substituents are independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, HO(C═O)—, (C₁-C₆)alkyl-O—(C═O)—, (C₁-C₆)alkyl-NH—(C═O)—, [(C₁-C₆)alkyl]₂-N—(C═O)—, phenyl-NH(C═O)—, phenyl-[((C₁-C₆)alkyl)-N]—(C═O)—, —NO₂, amino, (C₁-C₆)alkylamino, [(C₁-C₆)alkyl]₂amino, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, phenyl-HN—(C═O)—NH—, (phenyl-)₂N—(C═O)—NH—, phenyl-HN—(C═O)—[((C₁-C₆)alkyl)-N]—, (phenyl-)₂N—(C═O)—[((C₁-C₆)alkyl)-N]—, (C₁-C₆)alkyl-O—(C═O)—NH—, (C₁-C₆)alkyl-O—(C═O)—[((C₁-C₆)alkyl)-N]—, phenyl-O—(C═O)—NH—, phenyl-O—(C═O)—[((C₁-C₆)alkyl)-N]—, (C₁-C₆)alkyl-SO₂NH—, phenyl-SO₂NH—, (C₁-C₆)alkyl-SO₂—, phenyl-SO₂—, hydroxy, (C₁-C₆)alkoxy, perhalo(C₁-C₆)alkoxy, phenoxy, (C₁-C₆)alkyl-(C═O)—O—, phenyl-(C═O)—O—, H₂N—(C═O)—O—, (C₁-C₆)alkyl-HN—(C═O)—O—, [(C₁-C₆)alkyl-]₂N—(C═O)—O—, phenyl-HN—(C═O)—O—, (phenyl-)₂N—(C═O)—O—; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy.

21. A process according to claim 1, wherein R¹ is optionally substituted phenyl wherein said substituents are independently selected from the group consisting of halo, (C₁-C₆)alkyl, (C₂-C₆)alkenyl, perhalo(C₁-C₆)alkyl, —CN, (C₁-C₆)alkyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, hydroxy, (C₁-C₆)alkoxy, perhalo(C₁-C₆)alkoxy, (C₁-C₆)alkyl-(C═O)—O—, H₂N—(C═O)—O—, (C₁-C₆)alkyl-HN—(C═O)—O— and [(C₁-C₆)alkyl-]₂N—(C═O)—O—.

22. A process according to claim 1, wherein R¹ is optionally substituted phenyl containing two adjacent substituents which taken together with the carbon atoms to which they are attached form a five to six membered carbocyclic or heterocyclic ring.

23. A process according to claim 1, wherein R¹ is (R²)₂—N—, wherein each R¹ is independently selected from hydrogen, (C₁-C₆)alkyl, phenyl, (C₁-C₁₀)heterocyclic and (C₃-C₁₀)cycloalkyl; wherein each of the aforesaid R², (C₁-C₆)alkyl, phenyl, (C₁-C₁₀)heteroaryl, (C¹-C¹⁰)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂-, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-(C═O)—, (C₃-C₁₀)cycloalkyl-NH—(C═O)—, (C₁-C₆)alkyl-(C═O)—O—and phenyl-(C═O)—O—; wherein two R² (C₁-C₆)alkyl groups may be taken together with the nitrogen atom to form a five to six membered heterocyclic or heteroaryl ring.

24. A process according to claim 1, wherein R¹ is (R²)₂—N— and wherein each R² is independently selected from hydrogen, (C₁-C₄)alkyl, phenyl and (C₁-C₁₀)heterocyclic.

25. A process according to claim 1, wherein R⁴ is hydrogen.

26. A process for preparing a compound of the formula 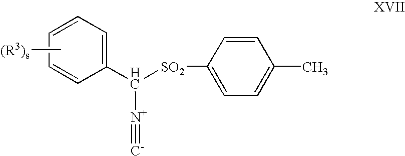
wherein each R³ is independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₈)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)— and (C₁-C₆)alkyl-(C═O)—O—; wherein two adjacent R³ substituents may be optionally taken together with the carbon atoms to which they are attached to form a five to six membered carbocyclic or heterocyclic ring;
s is an integer from zero to five;
or an acceptable salt thereof; comprising reacting a compound of the formula 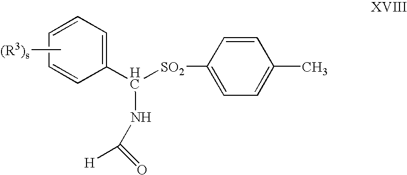
wherein R³ and s are as defined above, in the presence of POCl₃, 2,6-lutidine and a solvent.

27. A process according to claim 26, wherein said solvent is tetrahydrofuran.

28. A process according to claim 27, further comprising working up the reaction in the presence of citric acid.

29. A compound of the formula 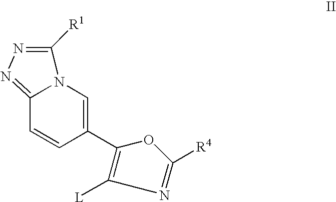
wherein L is bromo or chloro;
R¹ is selected from the group consisting of hydrogen, —C≡N, (C₁-C₆)alkyl, (C₂-C₆)alkenyl, (C₂-C₆)alkynyl, (C₃-C₁₀)cycloalkyl, phenyl, (C₁-C₁₀)heteroaryl, (C₁-C₁₀)heterocyclic and (R²)₂—N—; wherein each of the aforesaid (C₁-C₆)alkyl, (C₃-C₁₀)cycloalkyl, phenyl, (C₁-C₁₀)heteroaryl and (C₁-C₁₀)heterocyclic substituents may optionally be independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, —CN, (C₁-C₆)alkyl-(C═O)—, phenyl-(C═O)—, HO—(C═O)—, (C₁-C₆)alkyl-O—(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, —NO₂, amino, (C₁-C₆)alkylamino, [(C₁-C₆)alkyl]₂-amino, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, H₂N—(C═O)—NH—, (C₁-C₆)alkyl-HN—(C═O)—NH—, [(C₁-C₆)alkyl-]₂N—(C═O)—NH—, (C₁-C₆)alkyl-HN—(C═O)—[((C₁-C₆)alkyl)-N]—, [(C₁-C₆)alkyl-]₂N—(C═O)—[((C₁-C₆)alkyl)-N]—, phenyl-HN—(C═O)—NH—, (phenyl-)₂-N—(C═O)—NH—, phenyl-HN—(C═O)—[((C₁-C₆)alkyl)-N]—, (phenyl-)₂N—(C═O)—[((C₁-C₆)alkyl)-N]—, (C₁-C₆)alkyl-O—(C═O)—NH—, (C₁-C₆)alkyl-O—(C═O)—[((C₁-C₆)alkyl)-N]—, phenyl-O—(C═O)—NH—, phenyl-O—(C═O)—[((C₁-C₆)alkyl)-N]—, (C₁-C₆)alkyl-SO₂NH—, phenyl-SO₂NH—, (C₁-C₆)alkyl-SO₂—, phenyl-SO₂—, hydroxy, (C₁-C₆)alkoxy, perhalo(C₁-C₆)alkoxy, phenoxy, (C₁-C₆)alkyl-C═O)—O—, phenyl-(C═O)—O—, H₂N—(C═O)—O—, (C₁-C₆)alkyl-HN—(C═O)—O—, [(C₁-C₆)alkyl-₂N—C═O)-phenyl-HN—(C═O)—O—, (phenyl-)₂N—(C═O)—O—; wherein when said R¹ phenyl contains two adjacent substituents, such substituents may optionally be taken together with the carbon atoms to which they are attached to form a five to six membered carbocyclic or heterocyclic ring; wherein each of said moieties containing a phenyl alternative may optionally be substituted by one or two radicals independently selected from the group consisting of (C₁-C₆)alkyl, halo, (C₁-C₆)alkoxy, perhalo(C₁-C₆)alkyl and perhalo(C₁-C₆)alkoxy;
each R² is independently selected from hydrogen, (C₁-C₆)alkyl, phenyl, (C₁-C₁₀)heteroaryl, (C₁-C₁₀)heterocyclic and (C₃-C₁₀)cycloalkyl; wherein each of the aforesaid R¹ (C₁-C₆)alkyl, phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C ₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₁₀)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—; wherein two R² (C₁-C₆)alkyl groups may be taken together with the nitrogen atom to which they are attached to form a five to six membered heterocyclic or heteroaryl ring;
R⁴is selected from the group consisting of hydrogen, halo or R⁵—B—(CH₂)ₙ—;
n is an integer from zero to six;
each B is independently a bond, —(CHR⁶)—, —O—, —S—, —(SO₂)—, —(C═O)—, —O—(C═O)—, —(C═O)—O—, —(C═O)—NR⁶—, —(R⁶—N)—, —(R⁶—N)—SO₂—, —(R⁶—N)—(C═O)—, —SO₂—(NR⁶)—, —(R⁶—N)—(C═O)—(NR⁷)—, —(O)—(C═O)—(NR⁶)— or —(R⁶—N)—(C═O)—O—;
R⁵ is selected from the group consisting of hydrogen, —CF₃, —C═N, R⁹—(R⁸CH)ₘ—, phenyl, (C₁-C₁₀)heterocyclic, (C₁-C₁₀)heteroaryl, and (C₃-C₁₀)cycloalkyl; wherein each of the aforesaid R⁵ phenyl, (C₁-C₁₀)heteroaryl, (C₁-C₁₀)heterocyclic and (C₃-C₁₀)cycloalkyl substituents may optionally be substituted by one to four moieties independently selected from the group consisting of halo, (C₁-C₆)alkyl, (C₂-C₆)alkenyl, (C₂-C₈)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O—and phenyl-(C═O)—O—; wherein two adjacent R⁵ substituents of said phenyl, (C₁-C₁₀)heteroaryl, (C₁-C₁₀)heterocyclic and (C₃-C₁₀)cycloalkyl may optionally be taken together with the carbon or heteroatom to which they are attached to form a five or six membered carbocyclic or heterocyclic ring;
m is an integer from one to six;
R⁶ is hydrogen, (C₁-C₆)alkyl-SO₂— or (C₁-C₆)alkyl;
R⁷ is hydrogen or (C₁-C₆)alkyl;
each R⁸ is independently selected from the group consisting of hydrogen, amino, (C₁-C₆)alkoxy and (C₁-C₆)alkyl;
R⁹ is selected from the group consisting of hydrogen, (C₁-C₆)alkyl, (C₂-C₆)alkenyl, (C₂-C₆)alkynyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, phenyl-SO₂—NH—, (C₁-C₆)alkyl-SO₂—[((C₁-C₆)alkyl)-N]-, phenyl-SO₂—[((C₁-C₆)alkyl)-N]—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, (C₁-C₆)alkyl-(C═O)—O— and phenyl-(C═O)—O—;
or a salt thereof.

30. A compound of the formula 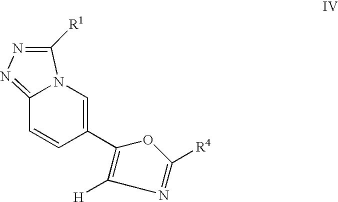
wherein R¹ and R⁴ are as defined above in claim 21; or a salt thereof.

31. A compound of the formula 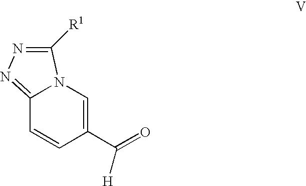
wherein R¹ is as defined above; or a salt thereof, wherein said compound is other than 3-isopropyl-[1,2,4]triazolo(4,3-a)-6-pyridinecarboxaldehyde.

32. A compound according to claim 22, wherein R¹ is (C₁-C₆)alkyl.

33. A compound according to claim 22, wherein R¹ is isopropyl.

34. A compound according to claim 22, wherein R⁴ is hydrogen.

35. A compound according to claim 22, wherein R⁴ is R⁵—B—(CH₂)ₙ— and n is zero.

36. A compound according to claim 22, wherein R⁴ is R⁵—B—(CH₂)ₙ— and n is an integer from one to five.

37. A compound according to claim 22, wherein R⁴ is R⁵—B—(CH₂)ₙ— is zero; B is a bond and R⁵ is selected from the group consisting of hydrogen, —CF₃, —C≡N, (C₁-C₁₀)heteroaryl, (C₁-C₁₀)heterocyclic or (C₃-C₁₀)cycloalkyl; wherein each of the aforesaid (C₁-C₁₀)heteroaryl, (C₁-C₁₀)heterocyclic and (C₃-C₁₀)cycloalkyl may optionally be substituted by one to three moieties independently selected from the group consisting of halo, (C₁-C₆)alkyl, (C₂-C₆)alkenyl, (C₁-C₆)alkynyl, perhalo(C₁-C₆)alkyl, hydroxy, (C₁-C₆)alkoxy, perhalo(C₁-C₆)alkoxy, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, —CN, (C₁-C₆)alkyl-(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)— and (C₁-C₆)alkyl-(C═O)—O—.

38. A compound according to claim 22, wherein R⁴ is R⁵—B—(CH₂)ₙ—; n is zero; B is —(C═O)—NR⁶—, —(R⁶—N)—, —(R⁶—N)—SO₂—, —(R⁶—N)—(C═O)—, >C═O—O—(C═O)—, —SO₂—(NR⁶)—, —(R⁶—N)(C═O)—(NR⁷)—; and
R⁵ is selected from the group consisting of hydrogen, (C₃-C₁₀)cycloalkyl or phenyl; wherein the aforesaid phenyl and (C₃-C₁₀)cycloalkyl may optionally be substituted by one to three moieties independently selected from the group consisting of halo, (C₁-C₆)alkyl, (C₂-C₆)alkenyl, (C₂-C₆)alkynyl, perhalo(C₁-C₆)alkyl, hydroxy, (C₁-C₆)alkoxy, perhalo(C₁-C₆)alkoxy, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[N(C₁-C₆)alkyl]—, —CN, (C₁-C₆)alkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)— and (C₁-C₆)alkyl-(C═O)—O—.

39. A compound according to claim 22, wherein R⁴ is R⁵—B—(CH₂)ₙ—; n is zero; B is —(C═O)—NR⁶—, —(R⁶—N)—, >C═O, —O—(C═O)—, —(R₆—N)—(C═O)— or —(R⁶—N)—(C═O)—(NR⁷)—; R⁹ is R⁹(R⁸CH)ₘ—; m is 1-6; R⁶ is hydrogen or methyl; R³ is hydrogen or methyl; and R⁹ is selected from the group consisting of hydrogen, (C₁-C₆)alkyl, (C₁-C₆)alkoxy, phenyl, (C₁-C₁₀)heteroaryl, (C₁-C₁₀)heterocyclic, (C₃-C₁₀)cycloalkyl, amino, (C₁-C₆)alkylamino, [(C₁-C₆)alkyl]₂amino, (C₁-C₆)alkyl-SO₂—NH—, phenyl-SO₂—NH—, (C₁-C₆)alkyl-SO₂—[N—(C₁-C₆)alkyl]-, phenyl-SO₂—[N—(C₁-C₆)alkyl]-, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂amino, (C₁-C₆)alkyl-SO₂—N—, (C₁-C₆)alkyl-(C═O)—NH—, (C₁-C₆)alkyl-(C═O)—[N(C₁-C₆)alkyl]-, phenyl-(C═O)—NH—, phenyl-(C═O)—[N—(C₁-C₆)alkyl]-, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, (C₁-C₁₀)heteroaryl-NH—(C═O)—, (C₁-C₁₀heterocyclic-NH—(C═O)—, (C₃-C₁₀)cycloalkyl-NH(C═O)—, HO—(C═O)—, (C₁-C₆)alkyl-O—(C═O)—, H₂N(C═O)—, (C₁-C₆)alkyl-NH—(C═O)—, [(C₁-C₆)alkyl]₂-N—(C═O)—, phenyl-NH—(C═O)—, phenyl-[N—((C₁-C₆)alkyl)]—(C═O)—, (C₁-C₆)alkyl-(C═O)—O— and phenyl-(C═O)—O—.

40. A compound according to claim 22, wherein R⁴ is R⁵—B—(CH₂)ₙ—; n is zero; B is —(R⁶—N)—; R⁵ is hydrogen or R⁹—(R⁸CH)ₘ—; m is 1-6; R⁶ is hydrogen or methyl; R⁸ is hydrogen or methyl; and R⁹ is selected from the group consisting of hydrogen, (C₁-C₆)alkyl, hydroxy, (C₁-C₆)alkoxy, amino, (C₁-C₆)alkylamino, [(C₁-C₆)alkyl]₂-amino, (C₂-C₆)alkenyl, (C₂-C₆)alkynyl, phenyl, (C₁-C₁₀)heteroaryl, (C₁-C₁₀)heterocyclic and (C₃-C₁₀)cycloalkyl.

41. A compound according to claim 22, wherein R⁴ is R⁵—B—(CH₂)ₙ—; n is one to four; B is —(C═O)—NR⁶—, —(R⁶—N)—, —(R⁶—N)—(C═O)— or —(R⁶—N)—(C═O)—(NR⁷)—; R⁵ is R⁹—(R⁸CH)ₘ—; m is 1-6; R⁶ is hydrogen or methyl; R⁸ is hydrogen or methyl; and R⁹ is selected from the group consisting of hydrogen, (C₁-C₆)alkyl, (C₁-C₆)alkoxy, phenyl, (C₁-C₁₀)heteroaryl, (C₁-C₁₀)heterocyclic, (C₃-C₁₀)cycloalkyl, amino, (C₁-C₆)alkylamino, [(C₁-C₆)alkyl]₂amino, (C₁-C₆)alkyl-SO₂—N H—, phenyl-SO₂—N H—, (C₁-C₆)alkyl-SO₂—[N—(C₁-C₆)alkyl]-, phenyl-SO₂—[N—(C₁-C₆)alkyl]-, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂-amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, (C₁-C₁₀)heteroaryl-NH—(C═O)—, (C₁-C₁₀)heterocyclic-NH—(C═O)—, (C₃-C₁₀)cycloalkyl-NH—(C═O)—, HO—(C═O)—, (C₁-C₆)alkyl-O—(C═O)—, H₂N(C═O)— (C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₆)alkyl-(C═O)—O— and phenyl-(C═O)—O—.

42. A compound according claim 1, wherein s is an integer from zero to four and each R³ is independently selected from the group consisting of halo, (C₁-C₆)alkyl, (C₂-C₆)alkenyl, (C₂-C₆)alkynyl, perhalo(C₁-C₆)alkyl, phenyl, (C₁-C₁₀)heteroaryl, (C₁-C₁₀)heterocyclic, (C₃-C₁₀)cycloalkyl, hydroxy, (C₁-C₆)alkoxy, perhalo(C₁-C₆)alkoxy, phenoxy, (C₁-C₁₀)heteroaryl-O—, (C₁-C₁₀)heterocyclic-O—, (C₃-C₁₀)cycloalkyl-O—, (C₁-C₆)alkyl-S—, (C₁-C₆)alkyl-SO₂—, (C₁-C₆)alkyl-NH—SO₂—, —NO₂, amino, (C₁-C₆)alkylamino, [(C₁-C₆)alkyl]₂—, amino, (C₁-C₆)alkyl-SO₂—NH—, (C₁-C₆)alkyl-(C═O)—NH—, (C₁-C₆)alkyl-(C═O)—[((C₁-C₆)alkyl)-N]—, phenyl-(C═O)—NH—, phenyl-(C═O)—[((C₁-C₆)alkyl)-N]—, —CN, (C₁-C₆)alkyl-(C═O)—, phenyl-(C═O)—, (C₁-C₁₀)heteroaryl-(C═O)—, (C₁-C₁₀)heterocyclic-(C═O)—, (C₃-C₁₀)cycloalkyl-(C═O)—, HO—(C═O)—, (C₁-C₆)alkyl-O—(C═O)—, H₂N(C═O)—(C₁-C₆)alkyl-NH—(C═O)—, [(C₁-C₆)alkyl]₂-N—(C═O)—, phenyl-NH—(C═O)—, phenyl-[((C₁-C₆)alkyl)-N]—(C═O)—, (C₁-C₁₀)heteroaryl-NH—(C═O)—, (C₁-C₁₀)heterocyclic-NH—(CO)—, (C₃-C₁₀)cycloalkyl-NH—(C═O)— and (C₁-C₆)alkyl-(C═O)—O—.

43. A compound according to claim 1, wherein s is an integer from zero to four and each R³ is independently selected from the group consisting of halo, —CN, (C₁-C₆)alkyl, (C₂-C₆)alkenyl, (C₂-C₆)alkynyl and perhalo(C₁-C₆)alkyl.

44. A compound according to claim 1, wherein s is an integer from zero to four and zero, one or two of R³ are independently selected from the group consisting of halo, (C₁-C₆)alkyl, perhalo(C₁-C₆)alkyl, hydroxy, (C₁-C₆)alkoxy, perhalo(C₁-C₆)alkoxy, amino, (C₁-C₆)alkylamino, [(C₁-C₆)alkyl]₂-amino, —CN, and H₂N(C═O)—.

45. A compound according to claim 1, wherein s is an integer from zero to three and each R³ is independently selected from the group consisting of halo, (C₁-C₆)alkyl, perhalo(C₁-C₆)alkyl, hydroxy, (C₁-C₆)alkoxy, perhalo(C₁-C₆)alkoxy, —NO₂, amino, (C₁-C₆)alkylamino, [(C₁-C₆)alkyl]₂-amino, —CN, and H₂N(C═O)—.

46. A compound according to claim 1, wherein s is an integer from zero to two and each R³ is independently selected from the group consisting of halo, (C₁-C₆)alkyl, perhalo(C₁-C₆)alkyl, (C₁-C₆)alkoxy, perhalo(C₁-C₆)alkoxy and —CN.

47. A compound according to claim 1, wherein s is an integer from zero to three and each R³ is independently selected from the group consisting of fluoro, chloro and methyl.

48. A compound selected from the group consisting of:
3-Isopropyl-6-[4-bromo-oxazol-5-yl]-[1,2,4]triazolo[4,3-a]pyridine; and
3-Isopropyl-6-[oxazol-5-yl]-[1,2,4]triazolo[4,3-a]pyridine; or
an acceptable salt thereof.
